(19) United States
(12) Patent Application Publication
(10) Pub. No.: US 2009/0233888 A1
(43) Pub. Date: Sep. 17, 2009

(54) TREATMENT OF DISEASE CONDITIONS THROUGH MODULATION OF HYDROGEN SULFIDE PRODUCED BY SMALL INTESTINAL BACTERIAL OVERGROWTH

(75) Inventor: Henry C. Lin, Albuquerque, NM (US)

Correspondence Address: DAVIS WRIGHT TREMAINE LLP/Los Angeles, 865 FIGUEROA STREET, SUITE 2400, LOS ANGELES, CA, 90017-2566, US

(73) Assignee: USC STEVENS, UNIVERSITY OF SOUTHERN CALIFORNIA, Los Angeles, CA (US)

(21) Appl. No.: 11/909,140

(22) PCT Filed: Mar. 23, 2006
(86) PCT No.: PCT/US06/10641

§ 371 Date: Sep. 19, 2007

Related U.S. Application Data
(60) Provisional application No. 60/664,599, filed on Mar. 23, 2005.

(51) Int. Cl. A61K 31/606; G01N 33/50; C12Q 1/02

(52) U.S. Cl. 514/166; 436/121; 436/119; 435/29

(57) ABSTRACT

The present invention relates to the treatment of a wide array of diseases and physiologic conditions based on modulating the level of hydrogen sulfide (H2S) in the body by at least partially eradicating small intestinal bacterial overgrowth (SIBO) in the gut. An H2S or lactulose breath test and/or detection of H2S or thiosulfate in the blood or urine may be used as a diagnostic and/or prognostic for assessing a systemic H2S load that exceeds a mammal's natural detoxification capacity. These tests may similarly be used to monitor the effectiveness of a therapeutic intervention for SIBO and/or the diseases or physiologic conditions whose pathology is linked thereto. Because SIBO is related to hyperhomocysteinemia, diseases and physiologic conditions that relate to hyperhomocysteinemia may further be monitored and treated in connection with the methods of the present invention.

### FIELD OF THE INVENTION

The invention relates to the treatment of various physiological conditions by modulating the level of hydrogen sulfide (H₂S) in the body.

### BACKGROUND OF THE INVENTION

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Hydrogen Sulfide (H₂S) is a colorless gas that, owing to its sulfur content, smells like rotten eggs. Frequently referred to as “sewer gas,” H₂S is highly poisonous—when inhaled, it has a level of toxicity similar to that of cyanide. H₂S inhibits aerobic respiration by binding reversibly to cytochrome oxidase and other metalloenzymes that are involved in aerobic cellular respiration (Dorman D C, Moulin F J M, McManus B E, Mahle K C, James R A, Struve M F. Cytochrome oxidase inhibition induced by acute hydrogen sulfide inhalation: correlation with tissue sulfide concentrations in the rat brain, liver, lung and nasal epithelium. Toxicological Sciences 65:18-25, 2002). Such inhibitory effect results in blockage of electron transfer within the mitochondria which in turn leads to respiratory arrest, loss of consciousness and death when exposed to H₂S at a high enough concentration (Costigan M G. Hydrogen sulfide: UK occupational exposure limits. Occup Environ Med 60:308-312, 2003). H₂S is found in petroleum and natural gas and is sometimes present in ground water. The odor associated with H₂S can be perceived at levels as low as 10 ppb (parts per billion). At levels of 50-100 ppm (parts per million), it may cause the human sense of smell to fail entirely. Low levels can cause eye irritation, dizziness, coughing, and headache. During strenuous exercise, inhalation of low levels of H₂S at 5 or 10 ppm is sufficient to shift from aerobic to anaerobic metabolism with increase in tissue lactic acid level (Bhambhani Y, Burnham R, Snydmiller G, et al. Comparative physiological responses of exercising men and women to 5 ppm hydrogen sulfide exposure. Am Ind Hyg Assoc J 55:1030-1035, 1994; Bhambhani Y, Burnham R, Snydmiller G, et al. Effects of 5 ppm hydrogen sulfide inhalation on biochemical properties of skeletal muscle in exercising men and women. Am Ind Hyg Assoc J 57:464-468, 1996; Bhambhani Y, Burnham R, Snydmiller G, et al. Effects of 10 ppm hydrogen sulfide inhalation on pulmonary function in healthy men and women. J Occup Env Med. 38:1012-1017, 1996; Bhambhani Y, Burnham R, Snydmiller G, et al. Effects of 10 ppm hydrogen sulfide inhalation on exercising men and women. J Occup Env Med 39:122-129, 1997). High levels (greater than approximately 600 ppm) can be fatal, typically due to respiratory failure and pulmonary edema.

Hydrogen is the major gas byproduct of bacterial fermentation, with as much as 12 liters per day being produced in the colon of normal subjects eating a typical diet. This gas is excreted as flatus and absorbed in the bloodstream to be exhaled in the breath or excreted through the skin. These routes of elimination of hydrogen are in addition to the metabolism of hydrogen primarily by one of two classes of hydrogen-consuming microbes in the gut. Methanogens use hydrogen to form methane, while sulfate-reducing bacteria use hydrogen to form H₂S. To generate energy to sustain life, sulfate-reducing bacteria use sulfate ion (So₄²⁻) as an oxidizing agent with the effect of reducing sulfate ion to hydrogen sulfide (H₂S) (see FIG. 3). This process depends on transmembrane multi-heme c-type cytochromes (Cyto. c₃). These two classes of bacteria compete for luminal hydrogen, and in a given individual, one class will usually dominate. Thus, a person who excretes hydrogen and methane would not be generating H₂S because methanogens out-compete sulfate-reducing bacteria. A third class of hydrogen-consumptive bacteria, the acetogenic bacteria, are found in a small percentage of humans and play only a small part in the utilization of intestinal hydrogen. H₂S is also generated by intestinal bacteria through the process of reduction of the sulfate to sulfide and metabolism of mucin and sulfur-containing amino acids such as methionine, homocysteine and cysteine.

In the body, H₂S must be detoxified by oxidation. While H₂S can be produced in large quantities by sulfate-reducing bacteria in the colon, it is normally rapidly metabolized by a specialized detoxification system in the colonic mucosa. More proximal sites of the gastrointestinal tract including the small intestine are much less efficient at detoxifying this gas. If the detoxification system were to be overwhelmed, H₂S would escape the gut to enter the portal vein. In the portal vein, a small amount of H₂S is detoxified by oxygen bound to hemoglobin. The majority would then enter the liver (see FIG. 4).

At sub-lethal levels of gas exposure, the biologic effects of H₂S are complex, as evidenced by a variety of clinical presentations. For example, after an accidental industrial exposure to this gas, a 24-year-old oil refinery worker complained of persistent fatigue, depression, anxiety, dizziness and trouble sleeping (K. H. Kilburn, Case report: profound neurobehavioral deficits in an oilfield worker overcome by hydrogen sulfide, Amer. J. Med. 1,306(5):301-305 (1993)). In other reports of similar industrial exposure to H₂S, impaired cognition with poor memory and difficulty with concentration were observed (C. Fenga et al., Cognitive sequelae of acute hydrogen sulphide poisoning. A case report, Medicina del Lavoro, 93(4):322-328 (2002)). Neurobehavioral abnormalities after environmental exposure to H₂S include impaired balance, loss of recall, irritability, tension, confusion, slow thinking, loss of libido, fatigue, lightheadedness, lack of concentration, decreased recent memory, disturbed sleep, dizziness, memory loss, shortness of breath, throat irritation, headache, long term memory loss, red and itching skin, cough, and wheezing (Kilburn K H. Effects of hydrogen sulfide on neurobehavioral function. S Med J 96(7):639-646, 2003). These adverse clinical effects of H₂S are supported by observations in animals. Rats exposed to ≧80 ppm H₂S have reduced spontaneous motor activity associated with poor spatial learning (M. F. Struve et al., Neurotoxicological effects associated with short-term exposure of Sprague-Dawley rats to hydrogen sulfide, Neurotoxicol., 22(3):375-385 (2001)) and memory (L. A. Partlo et al., Effects of repeated hydrogen sulphide (H₂S) exposure on learning and memory in the adult rat, Neurotoxicol., 22(2):177-189 (2001)). Correspondingly, demyelination of nerve fibers of the central nervous system has been observed with chronic exposure to H₂S in the environment (Sonyshikova T G. Demyelination of nerve fibers in the central nervous system caused by chronic exposure to natural hydrogen sulfide-containing gas. Bulletin of Experimental Biology and Medicine 136(4):328-332, 2003). Respiratory tract injury from inhaled H₂S includes olfactory neuronal loss, rhinitis bronchial epithelial hypertrophy and hyperplasia (Dorman D C, Struve M F, Gross E A, Brenneman K A. Respiratory tract toxicity of inhaled hydrogen sulfide in Fischer-344 rats, Sprague-Dawley rats and B6C3F1 mice following subchronic (90-day) exposure. Toxicology and Applied Pharmacology 198:29-39, 2004) accompanied by increased phlegm, shortness of breath and wheezing such as that seen in asthma (Madsen J, Sherson D, Kjoller H, Hansen I, Rasmussen K. Occupational asthma caused by sodium disulphite in Norwegian lobster fishing. Occupational and Environmental Medicine 61:873-874, 2004). Animals chronically exposed to H₂S have reduced body weight.

H₂S also has a beneficial and necessary role as a gaseous neuromodulator. Recent studies have found that endogenous H₂S is produced in the brain and the periphery. In humans, the pyridoxal-5′-phosphate-dependent enzymes cystathionine β-synthase (CBS) or cystathionine gamma lyase (CSE), can each catalyze the conversion of cysteine to H₂S and are important for the metabolism of sulfur-containing amino acids such as cystathionine, homocysteine and methionine (Du J B, Chen F R, Geng B, Jiang H F, Tang C S. Hydrogen sulfide as a messenger molecule in the cardiovascular system. J Peking Univ Health Sci 34:187, 2002). CBS and CSE are under negative feedback control by H₂S (see FIG. 5). CBS and CSE are also the enzymes involved in the metabolic clearance of homocysteine by the transsulfuration pathway (see FIG. 8). In heart tissues, H₂S is produced in part by 3-mercaptopyruvate sulfurtransferase. While CBS is found in the liver, kidneys and brain, CSE is found in the liver, kidneys, enterocytes and vascular smooth muscles. Thus, in the liver, endogenous H₂S production depends on both CBS and CSE. H₂S concentration in rat serum is ˜46 μM. At physiologic concentrations, H₂S has been identified to potentiate the NMDA (N-methyl-D-aspartate) receptor-mediated responses by inducing cyclic AMP (Kimura H. Hydrogen sulfide induces cyclic AMP and modulates the NADA receptor. Biochem Biophys Res Commun 267:129-133, 2000), protect neurons from oxidative stress as an endogenous reducing agent (Whiteman M, Armstrong J S, Chu S H, Siau J L, Wong B S, Cheung N S, Halliwell B, Moore P K. The novel neuromodulator hydrogen sulfide: an endogenous peroxynitrite ‘scavenger’. J Neurochem 90:765-768, 2004; Kimura Y, Kimura H. Hydrogen sulfide protects neurons from oxidative stress FASEB J (10): 1165-1167 (July 2004, epub May 2004)) induce calcium waves in astrocytes (Nagai Y, Tsugane M, Oka J I, Kimura H. Hydrogen sulfide induces calcium waves in astrocytes. FASEB J (3):557-559 (July 2004, epub May 2004)), and induce hippocampal long-term potentiation (LTP), a necessary part of learning and memory (Abe K, Kimura H. The possible role of hydrogen sulfide as an endogenous neuromodulator. J Neurosci 16:1066-1071, 1996). The importance of H₂S in cognitive function is evidenced by the finding that H₂S is severely decreased in the brain in patients with Alzheimer's disease accompanied by low levels of CBS activity, elevated level of homocysteine and reduced level of S-adenosylmethionine (SAM) which activates CBS (Eto K, Asada T, Arima K, Makifuchi T, Kimura H. Brain hydrogen sulfide is severly decreased in Alzheimer's disease. Biochem Biophys Res Commun 293:1485-1488, 2002). Excessive rather than reduced H₂S production is seen in Down's syndrome accompanied by overexpression of the H₂S synthesizing enzyme CBS (Kamoun P, Belardinelli M-C, Chabli A, Lallouchi K, Chadefaux-Vekemans B. Endogenous hydrogen sulfide overproduction in Down Syndrome. Am J Med Genetics 116A:310-311, 2003). H₂S has also been shown in an in vitro model to modulate the hypothalamus-pituitary-adrenal axis through inhibition of stimulated release of corticotropin-releasing hormone (CRH) from hypothalamus explants from rats (P. Navarra et al., Gaseous neuromodulators in the control of neuroendocrine stress axis, Annals NY Acad. Sci., 917:638-646 (2000)). In addition, H₂S has been shown to decrease blood pressure through its effect as a relaxant of vascular smooth muscle via K_{ATP} channels (Zhao W, Wang R. H₂S-induced vasorelaxation and underlying cellular and molecular mechanisms. Am J Physiol Heart Circ Physiol 283:H474-H480, 2002; Cheng Y, Ndisang J F, Tang G, Cao K, Wang R. Hydrogen sulfide-induced relaxation of resistance mesenteric artery beds of rats. Am J Physiol Heart Circ Physiol 287:H2316-H2323, 2004), and regulate hepatic circulatory pressure including portal pressure (Fiorucci S, Antonelli E, Mencarelli A, Orlandi S, Renga B, Rizzo G, Distrutti E, Shah V, Morelli A. The third gas: H₂S regulates perfusion pressure in both the isolated and perfused normal rat liver and in cirrhosis. Hepatology 42(3):539-548, 2005). H₂S is also a relaxant of the smooth muscles of the gastrointestinal tract (Hosoki R, Matsuki N, Kimura H. The possible role of hydrogen sulfide as an endogenous smooth muscle relaxant in synergy with nitric oxide. Biochem Biophys Res Commun 237:527-531, 1997; Teague B, Asiedu S, Moore P K. The smooth muscle relaxant effect of hydrogen sulphide in vitro: evidence for the physiological role to control intestinal contractility. Br J Pharmacol 137:139-145. 2002), uterine (Sidhu R, Singh M, Samir G, Carson R J. L-cysteine and sodium hydrosulphide inhibit spontaneous contractility in isolated pregnant rat uterine strips in vitro. Pharmacol Toxicol 2001, 88:198-203) and vas deferens. H₂S stimulates contractions of urinary bladder muscles via a neurogenic mechanism involving capsaicin-sensitive primary afferent nerves equipped with transient receptor vanilloid-1 receptors (TRPV 1) and efferent nerves acting on tachykinin 1 and tachykinin 2 receptors rather than acting via K_{ATP} channels (Patacchini R, Santicioli P, Giuliani S, Maggi C A. Hydrogen sulfide (H₂S) stimulates capsaicin-sensitive primary afferent neurons in the rat urinary bladder. Br J Pharmacol 142:31-34, 2004; Patacchini R, Santicioli P, Giuliani S, Maggi C A. Pharmacological investigation of the hydrogen sulfide (H₂S) contractile activity in rat detrusor muscle. Eur J Pharmacol 509:171-177, 2005; Trevisani M, Patacchini R, Nicoletti P, et al. Hydrogen sulfide causes vanilloid receptor 1-mediated neurogenic inflammation of the airways Br J Pharm 145(8):1123-32, 2005). Transient receptor potential vanilloid receptor-1 (TRPV1) also mediates H₂S induced neurogenic inflammation and atropine-resistant contractions of the airways via tachkinin 1 and tachykinin 2 receptor dependent pathways (Trevisani M, Patacchini R, Nicolletti P, Gatti R, Gazzieri D, Lissi N, Zagli G, Creminon C, Geppetti P, Harrison S. Hydrogen sulfide causes vanilloid receptor 1-mediated neurogenic inflammation of the airways. Br J Pharmacol 145(8):1123-31, 2005). The vasodilatory effects of H₂S may be beneficial in reducing ischemic myocardial pain and injury (Geng B, Yang J, Qi Y, Zhao J, Du Pang Y, Tang C. H₂S generated by heart in rat and its effects on cardiac function. Biochem Biophy Res Commun 313:362-368, 2004; Zunnunov Z R. Efficacy and safety of hydrogen sulfide balnerotherapy in ischemic heart disease in the arid zone. Terapevticheskii Arkhiv 76(8): 15-8, 2004) but may be responsible for hemorrhagic shock (Br J Pharmacol 143:881-889, 2004) and may be critical for avoiding spontaneous or essential hypertension (Yan H, Du J, Tang C. The possible role of hydrogen sulfide on the pathogenesis of spontaneous hypertension in rats. Biochem Biophys Res Comm 313:22-27, 2004), left ventricular hypertrophy (van Zwieten P A. Hydrogen sulphide: not only foul smelling but also pathophysiologically relevant. J Hypertension 21:1819-1820, 2003) or pulmonary hypertension (Zhang Q, Du J, Zhou W, Yan H, Tang C, Zhang C. Impact of hydrogen sulfide on carbon monoxide/heme oxygenase pathway in the pathogenesis of hypoxic pulmonary hypertension. Biochemical and Biophysical Res Comm 317:30-37, 2004). H₂S induces apoptosis of human aortic smooth muscles cells by activating caspase-3 (Yang G, Sun X F, Wang R. Hydrogen sulfide-induced apoptosis of human aorta smooth muscle cells via the activation of mitogen-activated protein kinases and caspase-3. FASEB J (14):1782-1784 (November 2004, epub September 2004)). In contrast, H₂S has also been reported to activate molecular pathways that lead to epithelial hyperplasia via Mitogen activated protein kinases (MAPK) mediated proliferative pathways (Deplancke B, Gaskins H R. Hydrogen sulfide induces serum-independent cell cycle entry in nontransformed rat intestinal epithelial cells. FASEB J (10):1310-312 (July 2003, epub May 2003)). H₂S is also reported to inhibit insulin secretion from pancreatic beta cells via stimulation of K_{ATP} channels (Yang W, Yang G, Jia X, Wu L, Wang R. Activation of K_{ATP} channels by H₂S in rat insulin-secreting cells and the underlying mechanisms. J Physiol 569(2):519-531, 2005). Increased H₂S production with increased activity of both CBS and CSE is seen in streptozotocin-induced diabetic rat (Yusuf M, Huat B T K, Hus A, Whiteman M, Bhatia M, Moore P K. Streptozotocin-induced diabetes in the rat is associated with enhanced tissue hydrogen sulfide biosynthesis. Biochem Biophys Res Comm 333:1146-1152, 2005). H₂S has a proinflammatory role in pancreatitis and its complications including lung injury (Bhatia M, Wong F L, Fu D, Lau H Y, Moochhala S M, Moore P K. Role of hydrogen sulfide in acute pancreatitis and associated lung injury. FASEB J (6):623-625 (April 2005, epub January 2005)). This proinflammatory role of H₂S is also important to septic shock and endotoxin-induced cardiovascular collapse (Li L, Bhatia M, Zhu Y Z, Zhu Y C, Ramnath R D, Wang Z J, Anuar F B M, Whiteman M, Salto-Tellez M, Moore P K. Hydrogen sulfide is a novel mediator of lipopolysaccharide-induced inflammation in the mouse. FASEB J (9): 1196-1198) as well as local tissue edema (Bhatia M, Sidhapuriwala J Moochhala S M, Moore P K. Hydrogen sulfide is a mediator of carrageenan-induced hind paw edema in the rat. Br J Pharmacol 145(2): 141-4, 2005) and inflammatory conditions of the colon and rectum such as ulcerative colitis and pouchitis (Ohge H, Fume J K, Springfield J, Rothenberger D A, Madoff R D, Levitt M D. Association between fecal hydrogen sulfide production and pouchitis. Dis Colon Rectum 48:469-475, 2005).

Homocysteine is a non-protein forming amino acid that is formed by demethylation of methionine, an essential amino acid obtained through the diet (see FIG. 6). Homocystine is an oxidized form of homocysteine. As used herein, the term “homocysteine” refers to both homocystine and homocysteine. There are two intermediates: S-adenosyl-methionine (SAM) and S-adenosyl-homocysteine (SAH) (see FIG. 6). Moreover, homocysteine is metabolized by two pathways: remethylation to methionine, or transsulfuration to cystathionine and then to cysteine. In the remethylation pathway, a methyl group from methyltetrahydrofolate (MTHF) is added in a step that is catalyzed by the enzymes methionine synthase (MS) and methylenetetrahydrofolate reductase (MTHFR). Remethylation requires the cofactors vitamin B12 and folate. In the liver, a significant portion of homocysteine is remethylated to methionine by betaine-homocysteine methyl transferase (BHMT) using methyl from betaine (see FIG. 7). Transsulfuration requires vitamin B6 and is catalyzed by CBS; the same enzyme that catalyzes the conversion of cysteine to hydrogen sulfide, as noted above (see FIG. 8).

Disruptions of the remethylation or transsulfuration pathways can result in an elevated plasma homocysteine level. While homocysteinuria is a rare genetic condition of severely elevated plasma homocysteine, mildly elevated plasma homocysteine (hyperhomocysteinemia) is relatively common and is associated with cardiovascular disease. Currently, hyperhomocysteinemia is generally explained on the basis of one or more of the following: (i) a mild inherited mutation that affects the efficiency of remethylation or transsulfuration of homocysteine, (ii) a nutritional deficiency of folate, vitamin B12 or vitamin B6, or (iii) hormonal changes, including a low estrogen level.

While hyperhomocysteinemia in the fasting state can occur with certain deficiencies in the remethylation pathway, detection of deficiencies in transsulfuration often requires methionine loading. This involves measuring plasma homocysteine after administration of methionine to shift metabolism toward transsulfuration. The frequency of patients having post-methionine load hyperhomocysteinemia exceeds the frequency of mutations in the CBS enzyme, indicating that non-genetic factors may contribute to mild deficiencies in the transsulfuration pathway. It has been proposed that depressed CBS activity may be due to metabolic down-regulation (V. Fonseca et al., Hyperhomocysteinemia and the endocrine system: implications for atherosclerosis and thrombosis, Endocrine Rev., 20(5):738-759 (1999)).

Methionine and cysteine are precursors of glutathione, the major intracellular molecule involved in defenses against free radicals. In the setting of decreased activity of CBS, while homocysteine accumulates, production of glutathione is reduced. These effects may result in a double hit of a low level of protective glutathione and high level of injurious homocysteine (J. L. Holzman, The role of low levels of the serum glutathione-dependent perioxidase and glutathione and high levels of serum homocysteine in the development of cardiovascular disease, Clin. Lab. 48(3-4):129-130 (2002)).

There is therefore a significant need in the art to identify a therapeutic method by which one can modulate the levels of H₂S in the body; particularly insofar as a harmful level of H₂S is based on escape of this gas from the gastrointestinal tract due to SIBO.

### SUMMARY OF THE INVENTION

The following embodiments and aspects thereof are described and illustrated in conjunction with compositions and methods which are meant to be exemplary and illustrative, not limiting in scope.

Various embodiments of the present invention relate to the treatment of a wide array of physiologic conditions in a mammal, including a number of diseases, the pathology of which relate to an elevated level of H₂S. In one embodiment of the present invention, a method is provided for treating such conditions and/or diseases by reducing the level of H₂S in the mammal. In one aspect of the invention, this may be accomplished by administering an agent or therapy that at least partially eradicates SIBO in the mammal; thereby reducing the level of H₂S in an amount sufficient to achieve beneficial results for the mammal with respect to a disease and/or physiologic condition.

Further embodiments of the present invention relate to the treatment of hyperhomocysteinemia and its related adverse biologic effects by identifying SIBO associated with H₂S production and/or by reducing the production of bacteria-derived H₂S. In the setting of SIBO, H₂S would be produced in the small intestine. Since the H₂S detoxifying capacity is limited in the small intestine, H₂S produced in the small intestine could escape detoxification to enter the liver. These adverse biologic effects may be mitigated or eliminated by at least partially eradicating SIBO.

Another embodiment of the present invention relates to the use of an H₂S or a lactulose breath test as a diagnostic or prognostic method or for assessing a systemic H₂S load that exceeds a mammal's natural detoxification capacity (both breath tests can be used to assess the severity of SIBO in a subject).

Another embodiment of the present invention relates to systemic detection and measurement of H₂S. The detection and measurement of H₂S may be performed by directly measuring H₂S concentration or by measuring thiosulfate as a marker of H₂S exposure in the blood. Thiosulfate may also be measured from urine. Optionally, a poorly digestible sugar (e.g., glucose, lactose, lactulose, xylose), or the poorly digestible sugar and methionine may be administered prior to the collection of blood and/or urine samples.

These particular embodiments of the present invention (i.e., an H₂S or lactulose breath test or systemic detection of H₂S or thiosulfate) may also be used to monitor the effectiveness of a therapeutic intervention for SIBO and/or any of the diseases or physiologic conditions whose pathology is linked thereto. This is based on the fact that successful treatment of SIBO may correlate with decreasing levels of H₂S in the body, outside the gastrointestinal tract.

The present invention also provides for kits for the diagnosis, prognosis, and/or treatment of disease conditions due to bacteria-derived H₂S. The kits are an assemblage of materials or components that facilitate in diagnosing, determining the prognosis and/or treating the disease conditions related to bacteria-derived H₂S. Instructions for use may also be included in the kits.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 illustrates the average breath hydrogen (H₂) profile during a lactulose breath test (LBT) in chronic fatigue syndrome (CFS) patients as compared to normal subjects and patients with IBS or fibromyalgia, in accordance with an embodiment of the present invention.

FIG. 2 illustrates the improvement of fatigue by eradication of SIBO, in accordance with an embodiment of the present invention.

FIG. 3 (prior art) illustrates the mechanism by which sulfate-reducing bacteria use hydrogen to form hydrogen sulfide, in accordance with an embodiment of the present invention. Sulfate-reducing bacteria use sulfate ion (SO₄²⁻) as an oxidizing agent with the effect of reducing a sulfate ion to hydrogen sulfide (H₂S). This process depends on transmembrane multi-heme c-type cytochromes (cyto.C3).

FIG. 4 illustrates the normal containment and loss of containment of gut microbes and microbial fermentation, in accordance with an embodiment of the present invention. Normally, gut microbes and microbial fermentation are primarily confined to the distal gut so that the colon is uniquely well equipped to protect itself and the human host with a detoxification system that oxidizes H₂S to thiosulfate. The cecum and right colon efficiently convert H₂S to thiosulfate, however the ileum has only 1/20^{th} of the rate of the cecum. In the setting of small intestine bacteria overgrowth (SIBO), there is a loss of containment of indigenous gut microbes. Abnormal microbial fermentation with production of bacteria-derived H₂S may take place more proximally including the small intestine where the detoxification capacity is limited.

FIG. 5 (prior art) illustrates the manner by which H₂S is formed by the actions of cystathionine β-synthase (CBS) or cysthionine gamma lyase (CSE), in accordance with an embodiment of the present invention. CBS and CSE are under negative feedback control by H₂S. CBS and CSE are also the enzymes involved in the metabolic clearance of homosysteine by the transsulfuration pathway.

FIG. 6 (prior art) illustrates the formation of homocysteine by demethylation of methionine, in accordance with an embodiment of the present invention. Additionally depicted are two intermediates, S-adenosyl-methionine (SAM) and S-adenosyl-homocysteine (SAH).

FIG. 7 (prior art) illustrates the remethylation pathway by which homocysteine is metabolized to methionine, in accordance with an embodiment of the present invention. In the remethylation pathway, a methyl group from methyltetrahydrofolate (MTHF) is added in a step that is catalyzed by the enzymes methionine snynthase (MS) and methylenetetrahydrofolate reductase (MTHFR). Remethylation requires the cofactors, vitamin B12 and folate. In an alternative pathway, betaine homocysteine methyltransferase (BHMT) converts homocysteine to methionine in a reaction which also converts betaine to dimethyl glycine.

FIG. 8 (prior art) illustrates the transsulfuration pathway by which homocysteine is metabolized to cystathionine and then to cysteine, in accordance with an embodiment of the present invention. Transsulfuration requires vitamine B6 and cystathionine β-synthase (CBS) which catalyzes the conversion of homocysteine and serine to cystathionine. This is the first reaction of the irreversible pathway for the catabolism of homocysteine. The enzyme cystathionine gamma lyase (CSE) catalyzes the conversion of cystathionine to cysteine. Transsulfuration takes place primarily in the liver, pancreas, small intestine and kidneys.

FIG. 9 illustrates the mechanism by which H₂S produced in the small intestine could escape detoxification to enter the liver, in accordance with an embodiment of the present invention. The presentation of bacteria-derived H₂S may interfere with hepatic transulfuration by exerting an inhibitory effect on CBS and CSE which may, in turn, impair homocysteine clearance leading to hyperhomocysteinemia.

FIG. 10 illustrates the effects of H₂S on intestinal transit in accordance with an embodiment of the present invention. Intestinal transit was slowed by fat in the distal ½ of gut as the ileal brake response (Buffer control: 53.77±5.96% vs. Ileal brake: 16.00±3.92%) (p<0.002). Hydrogen sulfide perfused in proximal compartment (H₂S Proximal) slowed transit when compared to Buffer control (37.78±3.80% vs. 53.77±5.96%) (p<0.016). Hydrogen sulfide perfused in distal compartment (H₂S Distal) did not slow transit when compared to Buffer control (60.72±8.97% vs. 53.77±5.96%)(p<0.57).

### DETAILED DESCRIPTION

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994); March, Advanced Organic Chemistry Reactions, Mechanisms and Structure 4th ed., J. Wiley & Sons (New York, N.Y. 1992), provides one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Beneficial results” may include, but are in no way limited to, lessening or alleviating the severity of the disease condition, preventing the disease condition from worsening, curing the disease condition and prolonging a patient's life or life expectancy. The disease conditions may relate to or may be modulated at least in part by H₂S.

“Conditions” and “disease conditions,” as used herein may include, but are in no way limited to pathological conditions, whether commonly recognized as diseases or not, that relate to or that are modulated by H₂S. Particular conditions and disease conditions that are believed to be appropriate to treat in connection with various embodiments of the present invention include conditions and disease conditions related, but are in no way limited to the following categories: Hypercoagulable states related to hyperhomocysteinemia (e.g., hyperhomocysteinemia, chronic renal failure, end stage renal disease, hemodialysis, peritoneal dialysis, vascular dementia, cardiovascular disease, stroke, cerebrovascular accidents, thrombotic disorder, hypercoagulable states, venous thrombosis, deep vein thrombosis, thrombophlebitis, thromboembolic disease, ischemic stroke, restenosis after percutaneous transluminal coronary angioplasty (PTCA), preeclampsia, vasculitis, digital ischemia, multifocal osteonecrosis, retinal vein occlusion, glaucoma, miscarriage, pregnancy complication, placental abruption, transplantation, diabetic retinopathy, ischemic bowel disease, cerebral vein thrombosis, atherosclerosis, coronary artery disease, penile venous thrombosis, impotence, central venous thrombosis, peripheral artery disease, intermittent claudication, hemorrhagic colitis, radiation enteritis and colitis, visceral ischemia, acute mesenteric ischemia, chronic mesenteric ischemia, hypertension, microangiopathy, macroangiopathy, recurrent leg ulcer, carotid stenosis, occlusive vascular disease, arterial aneurysm, abdominal aortic aneurysm); Vasodilatatory states (e.g., congestive heart failure, hepatopulmonary syndrome, high flow state associated with chronic liver disease, migraine headache, vascular headaches, dizziness, lightheadedness, orthostatic intolerance, postural hypotension, postural hypotension, postural orthostatic tachycardia syndrome, idiopathic pulmonary fibrosis, pulmonary hypertension, angioedema, vaso-vagal faints, neuroleptic malignant syndrome); Interference with function as neurotransmitter (e.g., learning disorder, learning disability, insomnia, dementia, age associated memory impairment, attention deficit/hyperactivity disorder (ADHD), mild cognitive impairment, Alzheimer's disease, Down's syndrome, autism, Parkinson's disease, depression, anxiety or anxiety disorder, Asperger syndrome); Interference with endocrine function (e.g., glucose intolerance, diabetes, reactive hypoglycemia, metabolic syndrome, low cortisol, hypothalamus-pituitary-adrenal dysfunction, myasthenia gravis syndrome, osteoporosis, autoimmune polyendocrine syndrome); Chronic pain syndromes due to stimulation of N-methyl-D-asparate (NMDA) receptors leading to hypersensitivity (e.g., chronic fatigue syndrome (CFS), central sensitivity syndrome, angina, syndrome X, chronic neck pain syndrome, chronic neuromuscular pain, osteoarthritis, muscle tension headaches, chronic headaches, cluster headache, temporalis tendonitis, sinusitis, atypical facial pain, trigeminal neuralgia, facial and neck pain syndrome, temperomandibular joint syndrome, idiopathic chronic low back pain, endometriosis, painful abdominal adhesions, chronic abdominal pain syndromes, coccydynia, pelvic floor myalgia (levator ani spasm), polymyositis, postherpetic neuralgia, polyradiculoneuropathies, mononeuritis multiplex, reflex sympathetic dystrophy, neuropathic pain, vulvar vestibulitis, vulvodynia, chronic regional pain syndrome, osteoarthritis, fibrositis, chronic visceral pain syndrome, female urethral syndrome, painful diverticular disease, functional dyspepsia, nonulcer dyspepsia, non-erosive esophageal reflux disease, acid-sensitive esophagus, interstitial cystitis, chronic pelvic pain syndrome, chronic urethral syndrome, chronic prostatitis, primary dysmenorrheal, dyspareunia, premenstrual syndrome (PMS), vulvodynia, ovarian remnant syndrome, ovulatory pain, pelvic congestion syndrome, myofasical pain syndrome, fibromyalgia polymyalgia rheumatica, Reiter's syndrome (reactive arthritis), rheumatoid arthritis, spondyloarthropathy, functional somatic syndromes, chronic regional pain syndromes, post polio syndrome, functional somatic syndrome); Injury to nasal and respiratory tract (e.g., rhinitis, asthma, multiple chemical sensitivity syndrome, reactive airway dysfunction syndrome, dysnomia, sick building syndrome, asthma, idiopathic pulmonary fibrosis, idiopathic pulmonary hypertension); Interference with visceral smooth muscle contractile function (e.g., dysphagia, gastroparesis, functional diarrhea, chronic constipation, defecation dysfunction, dysuria, atonic bladder, neurogenic bladder, irritable bowel syndrome (IBS), ileus, chronic idiopathic pseudoobstruction, Ogilvie's syndrome); Inhibition of aerobic metabolism/ischemia disorders (e.g., restless leg syndrome, chronic fatigue syndrome); Triggering of inflammation (e.g., immune dysfunction syndrome, multiple sclerosis (MS), eczema, psoriasis, atopic dermatitis, dermatitis, Crohn's disease, ulcerative colitis, ulcerative proctitis, pouchitis, nonspecific ulcerative colitis, inflammatory bowel disease (IBD), celiac disease, diversion colitis, collagenous colitis, lymphocytic colitis, blind loop syndrome, nonalcoholic steatohepatitis (NASH), fatty liver, chronic liver disease, cirrhosis, spontaneous bacterial peritonitis, postoperative ileus, systemic lupus erythematosis, mixed connective tissue disorder, undifferentiated connective tissue disorder, Raynaud's phenomenon, Kawasaki syndrome, polymyositis, dermatomyositis, myositis, multiple autoimmune syndrome, Sjögren's syndrome, lichen planus, idiopathic uveitis, gingivitis, stomatitis, otitis, necrotizing enterocolitis, intensive care unit (ICU) multiple organ failure, primary biliary cirrhosis, idiopathic myelofibrosis, polyarteritis nodosa, eosinophilic pleural effusion, eosinophilic gastroenteritis, eosinophilic esophagitis, graft vs. host disease, Grave's disease, idiopathic thyroid failure, Hashimoto's thyroiditis, autoimmune hepatitis, pancreatitis, CREST syndrome, autoimmune cholangitis, ankylosing spondylitis, atopic dermatitis, vitiligo, scleroderma, autoimmune ear disease, polyangiitis overlap syndrome, primary sclerosing cholangitis); overlap disorders (e.g., Gulf War syndrome, myalgic encephalomyelitis, food sensitivity, dysregulation spectrum syndrome, post-traumatic stress disorder (PTSD)); interference with regulation of apoptosis and proliferation (e.g., benign tumors, malignant tumors, cancer).

“Overlap disorder” or “overlap disorders” as used herein refers to two or more diseases or disease conditions that seem to share many common symptoms and often occur together. These disease or disease conditions include, for example, Gulf War syndrome, myalgic encephalomyelitis, food sensitivity, dysregulation spectrum syndrome, post-traumatic stress disorder (PTSD). Overlap disorders may also be commonly termed as “overlap syndromes,” “central sensitivity syndromes,” and “dysregulation syndromes.”

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

“Treatment” and “treating,” as used herein refer to both therapeutic treatment and prophylactic or preventative measures, wherein the object is to prevent or slow down (lessen) the targeted condition, disease or disorder even if the treatment is ultimately unsuccessful. Those in need of treatment include those already with the disorder as well as those prone to have the disorder or those in whom the disorder is to be prevented.

In one embodiment of the present invention, a condition, disease or disease condition in a mammal may be treated by at least partially eradicating small intestinal bacterial overgrowth (SIBO) to reduce the levels of H₂S in the body. While not wishing to be bound by any particular theory, it is believed that this may relate to the treatment of hyperhomocysteinemia caused by elevated levels of H₂S.

Indeed, one of the central features of the present invention is the treatment of hyperhomocysteinemia and its related adverse biologic effects by the identification of SIBO associated with H₂S production and/or by reducing the production of bacteria-derived H₂S. While not wishing to be bound by any particular theory, the inventor believes that the presentation of bacteria-derived H₂S interferes with hepatic transsulfuration by exerting an inhibitory effect on CBS and CSE which may, in turn, impair homocysteine clearance leading to hyperhomocysteinemia. The inventor further believes that exposure to bacteria-derived H₂S interferes with physiologic functions of endogenous H₂S.

In the liver, bacteria-derived H₂S may interfere with the metabolic pathways of the host. The liver would then use oxidation of H₂S to thiosulfate as the primary detoxification strategy. Any H₂S that escapes hepatic detoxification could then be transported throughout the body via the systemic circulation, to the muscles where it may interfere with aerobic metabolism but could be oxidized by oxygen bound to myoglobin and finally, to kidney where it could be excreted as thiosulfate. An elevated level of plasma H₂S or thiosulfate would indicate exposure to excessive H₂S. Thus, in the setting of small intestinal bacterial overgrowth (SIBO) where the small bowel may be exposed to large amounts of H₂S, a significant amount of this gas may be absorbed into the bloodstream. Here, while some H₂S may be detoxified by oxygen bound to hemoglobin and a further small amount may be detoxified in muscles where it is oxidized by oxygen bound to myoglobin, any remaining amount will be transported throughout the body including the lungs, where it may be exhaled in the breath. The excretion of H₂S in the breath should then be a marker for a systemic load which exceeds the detoxification capacity.

While it is not known what level of exposure to H₂S may occur in SIBO, the inventor's preliminary data showed an average peak hydrogen concentration of 85 ppm in the exhaled breath of patients with chronic fatigue syndrome (CFS), demonstrating the availability of considerable gas substrate for the sulfate-reducing bacteria. The inventor thus hypothesized that exposure to bacteria-derived H₂S may interfere with physiologic functions that are normally controlled by endogenous H₂S.

In short, the enzyme that is critical for removing circulating homocysteine from the body (CBS) also makes H₂S in the brain. Furthermore, this enzyme operates by negative feedback. In other words, when H₂S escapes the gastrointestinal tract (e.g., due to SIBO) and enters the bloodstream, it may decrease the level of activity of CBS; thereby resulting in two significant physiological problems: (1) an inhibition of the endogenous production of H₂S in the brain, where the molecule acts as a necessary gaseous neuromodulator, and (2) potentially toxic levels of H₂S in other parts of the body where it results in consequences ranging from moderately detrimental to quite serious. In addition, inhibition of CBS by bacteria-derived H₂S reduces transsulfuration to impair metabolism of homocysteine. Hyperhomocysteinemia may be a consequence of this action of bacteria-derived H₂S. There are a great number of physiologic conditions whose pathology can be traced to one of the aforementioned effects of increased H₂S.

The direct and indirect (homocysteine) effect of H₂S may account for many of the symptoms and findings of CFS patients including impaired postural cardiovascular response, impaired cognition, muscle fatigue (shift from aerobic to anaerobic metabolism) and disturbances of the HPA axis. The present invention offers a significant advance in the management of hyperhomocysteinemia, because genetic explanations such as the alanine/valine (A/V) gene polymorphism of 5,10-methylenetetrahydrofolate reductase (i.e., the “MTHFR VV genotype”) only account for a small number of patients, normalization of homocysteine level is rarely achieved using even high doses of vitamins (U. Poge et al., Intravenous treatment of hyperhomocysteinemia in patients with chronic hemodialysis—a pilot study, Renal Failure, 26(6):703-708 (2004)), and folate deficiency is not the cause of hyperhomocysteinemia in end-stage renal disease (C. van Guldener et al., Homocysteine metabolism in renal failure, Kidney Int., 59 (Supp. 78):S234-37 (2001)).

These issues are exemplified by the problem of chronic renal disease patients. The most commonly reported symptoms in these patients are fatigue, disturbed sleep, abdominal bloating and gas, muscle cramps, and bad taste in mouth (M. V. Rocco et al., Cross-sectional study of quality of life and symptoms in chronic renal disease patients: the modification of diet in renal disease study, Amer. J. Kidney Dis., 29(6):888-896 (1997)). The complaint of chronic bloating in these patients can not be explained by delayed gastric emptying W. E. Soffer et al., Gastric emptying in chronic renal failure patients on hemodialysis, J. Clin. Gastroenterol., 9(6):651-653 (1987)). In 1998, there were 320,000 patients on dialysis, 13.3 million patients with mild-severe decrease in glomerular filtration rate and another 5.9 million patients with chronic kidney disease without a reduction in the glomerular filtration rate (M. J. Sarnak et al., Kidney disease as a risk factor for development of cardiovascular disease, Circ. 108:2154-2169 (2003)). The prevalence of coronary artery disease is elevated in all of these patients and is a leading cause of mortality and morbidity (R. N. Foley et al., Clinical epidemiology of cardiovascular disease in chronic renal disease, Amer. J. Kidney Dis. 32(Supp. 3): 112-9 (1998)). In hemodialysis patients, the prevalence of cardiovascular disease is thought to be 40% with an annual rate of cardiovascular events of 9% (US Renal Data System 1992, Annual Report IV, Comorbid conditions and correlations with mortality risk among 3,399 incident hemodialysis patients, Amer. J. Kidney Dis., 20 (Supp 2):32-8 (1992)). Hyperhomocysteinemia is a well known risk factor for cardiovascular complications in chronic kidney diseases with plasma homocysteine level reaching 100 μM/L or higher when the glomerular filtration rate drops below 70 ml/min (A. Moustapha et al., Prospective study of hyperhomocysteinemia as an adverse cardiovascular risk factor in end-stage renal disease, Circ. 97:138-141 (1998)). Hyperhomocysteinemia was observed in all hemodialysis patients and in 95% of peritoneal dialysis patients (P. G. Chiarello et al., Hyperhomocysteinemia and oxidative stress during dialysis treatment, Renal Failure, 25(2):203-213 (2003)). The mean homocysteine level in hemodialysis patients is gender specific, with a higher mean value in males of 66.8 vs. 40.6 in females (C. Libetta et al., Prevalence of hyperhomocysteinemia in male hemodialysis patients, Kidney Int'l., 64(4): 1531 (2003)). The cause of this hyperhomocysteinemia was heretofore unknown, although it is not considered to be explained by uremic retention alone (A. Perna et al., Homocysteine in Uremia, Amer. J. Kidney Dis., 41(3):S123-S126 (2003)). Hyperhomocysteinemia is also associated with carotid atherosclerosis in peritoneal dialysis patients (T. Ohkuma et al., C-reactive protein, lipoprotein(a), homocysteine, and male sex contribute to carotid atherosclerosis in peritoneal dialysis patients, Amer. J. Kidney Dis., 42(20):355-61 (2003)). Possible mechanisms for the toxicity of homocysteine include oxidative stress through reactive oxygen species, nitric oxide binding, production of homocysteineylated/acylated proteins, accumulation of the precursor of homocysteine or S-adenosyl-methionine which inhibits transmethylation (A. Perna et al., Possible mechanisms of homocysteine toxicity, Kidney Int'l-Supp., 63(Supp. 84):S137-S140 (2003)).

Furthermore, recent data suggests that antibiotics may be effective in the treatment of vascular disease; wherein changes in coronary flow velocity reserve (CFVR) were negatively correlated with changes in high-sensitivity C-reactive protein levels in patients receiving antibiotic therapy (E. Hyodo et al., Effect of azithromycin therapy on coronary circulation in patients with coronary artery disease, Amer. J. Cardiol. 94(11): 1426-1429, and H. B. Leu et al., Risk stratification and prognostic implication of plasma biomarkers in nondiabetic patients with stable coronary artery disease: the role of high sensitivity C-reactive protein, Chest 126(4):1032-1039 (2004)). These observations and other findings suggesting chronic inflammation are likely to be consequences of SIBO, once again suggesting the therapeutic potential of an embodiment of the present invention drawn to treating vascular disease by at least partially eradicating SIBO, as well as an embodiment of the present invention relating to the diagnostic detection and treatment of the cause of hyperhomocysteinemia and elevated C-reactive protein to reduce their associated complications.

Restless leg syndrome is yet another disease condition for which H₂S may be the cause or a contributing cause. While not wishing to be bound by any particular theory, the inventor believes that H₂S blocks aerobic metabolism in muscles to result in lactate build up in leg muscles that drive the movement.

At least partially eradicating the bacterial overgrowth may be accomplished by any suitable method, as will be recognized and readily implemented by those of skill in the art. U.S. Pat. No. 6,861,053, which is incorporated by reference herein in its entirety, describes a number of techniques for at least partially eradicating SIBO. This may be accomplished by, for example, administering an antimicrobial agent, including but not limited to a natural, synthetic, or semi-synthetic antibiotic agent; for example, a course of antibiotics such as, but not limited to, neomycin, metronidazole, teicoplanin, doxycycline, tetracycline, norfloxacin, ciprofloxacin, augmentin, cephalexin (e.g., Keflex), penicillin, ampicillin, kanamycin, rifamycin, rifaximin or vancomycin, each of which may be administered orally, intravenously, or rectally.

Alternatively, an antimicrobial chemotherapeutic agent, such as a 4- or 5-aminosalicylate compound may be used to at least partially eradicate SIBO. These can be formulated for ingestive, colonic, or topical non-systemic delivery systems or for any systemic delivery systems. Commercially available preparations include 4-(p)-aminosalicylic acid (i.e., 4-ASA or para-aminosalicylic acid) or 4-(p)-aminosalicylate sodium salt. 5-aminosalicylates have antimicrobial, as well as anti-inflammatory properties, in useful preparations including 5-aminosalicylic acid (i.e., 5-ASA, mesalamine, or mesalazine) and conjugated derivatives thereof, available in various pharmaceutical preparations such as Asacol, Rowasa, Claversal, Pentasa, Salofalk, Dipentum (olsalazine), Azulfidine (SAZ; sulphasalazine), ipsalazine, salicylazobenzoic acid, balsalazide, or conjugated bile acids, such as ursodeoxycholic acid-5-aminosalicylic acid, and others.

Another method of at least partially eradicating SIBO, particularly useful when a subject does not respond well to oral or intravenous antibiotics or other antimicrobial agents alone, is administering an intestinal lavage or enema, for example, small bowel irrigation with a balanced hypertonic electrolyte solution, such as Go-lytely or fleet phosphosoda preparations. The lavage or enema solution is optionally combined with one or more antibiotic(s) or other antimicrobial agent(s).

Another method of at least partially eradication SIBO, particularly useful when a subject does not respond well to oral or intravenous antibiotics or other antimicrobial agents alone, is administering a bismuth-containing compound such as bismuth subsalicylate as exemplified by Pepto-bismol.

Another method of at least partially eradicating SIBO, particularly useful when a subject does not respond well to oral or intravenous antibiotics or other antimicrobial agents alone, is administering compounds that bind iron in the intestinal lumen to reduce the availability of this critical micronutrient that is needed by bacteria for survival such as lactoferrin, activated lactoferrin, colostrom, transferring, egg white lysozyme, lactoferricin, hydrolyzed whey powder, iron binding proteins, ferritin, transferrin. These agents have an antimicrobial effect.

Another strategy is to administer compounds that bind hydrogen sulfide (Mitsui T, Edmond L M, Magee E A, Cummings J H. The effects of bismuth, iron, zinc and nitrates on free sulfide in batch cultures seeded with fecal flora. Clinica Chimica Acta 335:131-135, 2003) produced by SIBO including nitrates, iron, zinc and bismuth. Iron and zinc are common nutritional supplements. Nitrates are found in processed meats and can also be taken as a supplement. Bismuth is readily available in the form of bismuth subsalicylate (e.g., Pepto-bismo).

Another method of at least partially eradicating SIBO employs a probiotic agent, for example, an inoculum of a lactic acid bacterium or bifidobacterium. The inoculum is delivered in a pharmaceutically acceptable ingestible formulation, such as in a capsule, or for some subjects, consuming a food supplemented with the inoculum is effective, for example a milk, yogurt, cheese, meat or other fermentable food preparation. Useful probiotic agents include Bifidobacterium sp. or Lactobacillus species or strains, e.g., L. acidophilus, L. rhamnosus, L. plantarum, L. reuteri, L. paracasei subsp. paracasei, L. casei Shirota, L. salivarius or B. infantis (L. O'Mahony et al., Lactobacillus and Bifidobacterium in irritable bowel syndrome: symptom responses and relationship to cytokine profiles, Gastroenterol., 128:541-551 (2005)).

Furthermore, because methanogens are known to outcompete sulfur-reducing bacteria in vivo, in one embodiment of the present invention, methanogens may be used in connection with a therapeutic for the problems associated with bacteria-derived H₂S.

Optionally, after at least partial eradication of SIBO, use of antimicrobial agents or probiotic agents can be continued to prevent further development or relapse of SIBO.

Another method of at least partially eradicating SIBO is by normalizing or increasing phase III interdigestive intestinal motility with any of several modalities to at least partially eradicate the bacterial overgrowth, for example, by suitably modifying the subject's diet to increase small intestinal motility to a normal level (e.g., by increasing dietary fiber), or by administration of a chemical prokinetic agent to the subject, including bile acid replacement therapy when this is indicated by low or otherwise deficient bile acid production in the subject.

For purposes of the present invention, a prokinetic agent is any chemical that causes an increase in phase III of interdigestive motility of a human subject's intestinal tract. Increasing intestinal motility, for example, by administration of a chemical prokinetic agent, prevents relapse of the SIBO condition, which otherwise may recur within about two months, due to continuing intestinal dysmotility. The prokinetic agent causes an in increase in phase III of interdigestive motility of the human subject's intestinal tract, thus preventing a recurrence of the bacterial overgrowth. Continued administration of a prokinetic agent to enhance a subject's phase III of interdigestive motility can extend for an indefinite period as needed to prevent relapse of the SIBO condition.

The prokinetic agent may be a known prokinetic peptide, such as motilin, or a functional analog thereof, such as a macrolide compound, for example, erythromycin (50 mg/day to 2000 mg/day in divided doses orally or I.V. in divided doses), or azithromycin (250-1000 mg/day orally). In addition, a 5-hydroxytryptamine (HT or serotonin) receptor directed drug such as tegaserod, a 5-HT4 receptor agonist, may be used to induce phase III of interdigestive motility. Other agents with prokinetic activities include 5-HT receptor antagonist, such as ondansetron (2-4 mg up to every 4-8 hours I.V.; pediatric 0.1 mg/kg/day), cilansetron, granisetron or alosetron may also be used.

Additionally, a bile acid, or a bile salt derived therefrom, is another suitable prokinetic agent for inducing or increasing phase III of interdigestive motility. Useful bile acids include, but are not limited to, ursodeoxycholic acid and chenodeoxycholic acid; useful bile salts include sodium or potassium salts of ursodeoxycholate or chenodeoxycholate, or derivatives thereof.

A compound with cholinergic activity, such as cisapride (i.e., Propulsid; 1 to 20 mg, one to four times per day orally or I.V.), may also be used as a prokinetic agent for inducing or increasing phase III of interdigestive motility.

A dopamine antagonist, such as metoclopramide (1-10 mg four to six times per day orally or I.V.), domperidone (10 mg, one to four times per day orally), or bethanechol (5 mg/day to 50 mg every 3-4 hours orally; 5-10 mg four times daily subcutaneously), octreotide or cholecystonin or its analogues may also be used in accordance with an alternate embodiment of the present invention for inducing or increasing phase III interdigestive motility.

A nitric oxide altering agent, such as nitroglycerin, nomega-nitro-L-arginine methylester (L-NAME), or N-monomethyl-L-arginine (L-NMMA) may also be used.

An antihistamine, such as promethazine (oral or I.V. 12.5 mg/day to 25 mg every four hours orally or I.V.), meclizine (oral 50 mg/day to 100 mg four times per day), or certain other antihistamines, may also be used as prokinetic agents for inducing or increasing phase III of interdigestive motility.

In an alternate embodiment, neuroleptic agents may also be used, including prochlorperazine (2.5 mg/day to 10 mg every three hours orally; 25 mg twice daily rectally; 5 mg/day to 10 mg every three hours, not to exceed 240 mg/day intramuscularly; 2.5 mg/day to 10 mg every four hours I.V.), chlorpromazine (0.25 mg/lb. up to every four hours [5-400 mg/day] orally; 0.5 mg/lb. up to every 6 hours rectally; intramuscular 0.25/lb. every six hours, not to exceed 75/mg/day), or haloperidol (oral 5-10 mg/day orally; 0.5-10 mg/day I.V.). Also useful as a prokinetic agent, for purposes of the present invention, is a kappa agonist, such as fedotozine (1-30 mg/day), but not excluding other opiate agonists.

The preceding are merely illustrative of some suitable methodologies by which SIBO can be at least partially eradicated in accordance with alternate embodiments of the present invention to treat a disease or condition or combination of diseases and conditions in a mammal. These can be used separately or in combination by the practitioner as suits the needs of an individual mammalian subject, and as is effective in treating the targeted disease or condition to seek beneficial results.

The therapeutic agents according to the invention may be delivered in a therapeutically effective amount. The precise therapeutically effective amount is that amount of the agent that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of an agent and adjusting the dosage accordingly. For additional guidance, see Remington: The Science and Practice of Pharmacy (Gennaro ed. 20th edition, Williams & Wilkins Pa., USA) (2000).

To achieve the goal of eradication of SIBO, antibiotics may be used. For example, rifaximin is a poorly absorbed antibiotic that requires bile salts for solubility. Its bioavailability is therefore limited to the small intestine with sparing of the colonic flora. This agent is also not associated with plamid transfer of resistance by targeted microorganisms. Typical dose of rifaximin to achieve about 60-70% efficacy in achieving successful partial eradication of SIBO is about 400 mg three times a day (TID) for about 10 days. Alternatively, an elemental diet may be used such as Vivonex Plus® taken at the dose of 1 packet mixed with water for breakfast, 2 packets for lunch and 2 packets for dinner. Lactoferrin may be taken at the dose of from about 250 to about 500 mg once to three times per day. Pepto-bismol® may be taken at the dose of about 60 ml about every 6 hours for about 48 hours as a liquid with about 262 mg bismuth subsalicylate in about 15 ml. Once successful partial eradication of SIBO is achieved, the time to relapse of SIBO may be delayed with a prokinetic agent such as erythrymycin. About 50 mg of erythrymycin may be given as ¼ tsp of EES-200 pediatric elixir at bedtime or about 2 mg of tegaserod may be given at bedtime. Alternatively, a probiotic may be used such as B. infantis given as one capsule in the morning (Align®). Typical dosages of an effective amount of a therapeutic agent can be as indicated to the skilled artisan by the in vitro responses or responses in animal models. Such dosages typically can be reduced by up to about one order of magnitude in concentration or amount without losing the relevant biological activity. Thus, the actual dosage will depend upon the judgment of the physician, the condition of the patient, and the effectiveness of the therapeutic method based, for example, the responses observed in the appropriate animal models, as previously described.

Another embodiment of the present invention relates to the use of an H₂S or a lactulose breath test as a diagnostic and/or prognostic method for assessing a systemic H₂S load that exceeds a mammal's natural detoxification capacity (both breath tests can be used to assess the severity of SIBO in a subject). Bacteria-derived H₂S may be detected in the exhaled breath using gas analyzers sensitive to sulfur-containing compounds. H₂S concentration in exhaled breath may be measured using a total/species sulfur analyzer.

Another embodiment of the present invention relates to systemic detection and measurement of H₂S. The detection and measurement of H₂S may be performed by directly measuring H₂S concentration or by measuring thiosulfate as a marker of H₂S exposure in the blood. The thiosulfate may also be measured from urine. A poorly digestible sugar (e.g., glucose, lactose, lactulose, xylose), or a poorly digestible sugar and methionine may be administered prior to the collection of blood and/or urine samples. A poorly digestible sugar is one which there is a relative or absolute lack of capacity in a human for absorption thereof or for enzymatic degradation or catabolism thereof. While not wishing to be bound by any particular theory, the inventor believes that that CFS may depend on a shift in host-gut microbial relationship with abnormal exposure to H₂S as a consequence of small intestinal bacterial overgrowth. In SIBO, there is an abnormal expansion of the gut microbial population into the small intestine, a region of the gut where fermentable substrates are readily available to result in increased microbial gas production including H₂S. The exposure of the host to this toxic gas is reduced by an intestinal detoxification system that converts H₂S to the stable metabolite, thiosulfate by oxidation (1) according to the following:

4S²⁻+3O₂→2S₂O₃²⁻

While H₂S detoxification is very effective in the colon, the H₂S detoxification capacity of the small intestine is only 1/20th that of the colon, an area that would be exposed to H₂S in SIBO. In that setting, some H₂S may escape conversion to thiosulfate to appear in the systemic circulation. An elevated concentration of H₂S in systemic blood would support abnormal systemic exposure to this toxic gas. Regardless of the site of detoxification, H₂S that is converted to thiosulfate would enter the portal circulation from the intestine. Accordingly, either an elevated thiosulfate concentration in portal blood or an elevated H₂S concentration in systemic blood would provide evidence for abnormal exposure to this toxic gas.

These particular embodiments of the present invention (i.e., an H₂S or lactulose breath test or systemic detection of H₂S or thiosulfate) may also be used to monitor the effectiveness of a therapeutic intervention for SIBO and/or any of the diseases or physiologic conditions whose pathology is linked thereto. This is based on the fact that successful treatment of SIBO may correlate with decreasing levels of H₂S in the body, outside the gastrointestinal tract.

The present invention is also directed to kits for diagnosing, determining a prognosis and/or treating a disease condition related to bacteria-derived H₂S. The kits are an assemblage of materials or components that facilitate diagnosing, determining the prognosis, and/or treating the disease condition related to bacteria-derived H₂S.

The exact nature of the components configured in the inventive kit depends on its intended purpose. For example, some embodiments are configured for the purpose of diagnosing and/or determining the prognosis of a disease condition by detecting the presence and/or concentration of bacteria-derived H₂S. In these embodiments, the kit may contain an air-tight breath sampling container, an air-tight blood sampling container, a urine sampling container and/or a quantity of a poorly digestible sugar. In other embodiments, the kit is configured for treating a disease condition by at least partially eradicating SIBO. In these embodiments, the kit may contain a therapeutic agent for at least partially eradicating SIBO; for example, a antimicrobial agent, a probiotic agent and/or a prokinetic agent.

Instructions for use may be included in the kit. “Instructions for use” typically include a tangible expression describing the technique to be employed in using the components of the kit to effect a desired outcome, such as to detect the presence of H₂S or thiosulfate to diagnose or determine a prognosis of a disease condition related to bacteria-derived H₂S, or to at least partially eradicating SIBO to treat the disease condition related to bacteria-derived H₂S. For example, the kit may include instructions to administer a poorly digestible sugar to the mammal and to obtain breath, blood and/or urine samples and instructions to analyze the samples to detect the presence and/or concentration of H₂S and/or thiosulfate. Kits for treating the disease condition may include instructions to administer a therapeutic agent to at least partially eradicate the bacterial overgrowth. Instructions for use may also include instructions on how to use the kit to corroborate a suspected diagnosis of a disease condition with the results obtained from using the kit. Optionally, the kit also contains other useful components, such as, diluents, buffers, pharmaceutically acceptable carriers, syringes, catheters, applicators, pipetting or measuring tools, sampling containers or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit. The packaging material is constructed by well known methods, preferably to provide a sterile, contaminant-free environment. As used herein, the term “package” refers to a suitable solid matrix or material such as glass, plastic, paper, foil, and the like, capable of holding the individual kit components. The packaging material generally has an external label which indicates the contents and/or purpose of the kit and/or its components.

### EXAMPLES

The following Examples are illustrative of the relationship among SIBO, elevated levels of H₂S, and particular conditions that may be treated in accordance with various embodiments of the present invention. The methods of the present invention have uses beyond those illustrated herein, however; for instance, as described above in connection with a wide range of diseases and conditions. These Examples are therefore in no way intended to delineate the extent to which the invention may find application in connection with alternate diseases and conditions.

### Example 1

### Chronic Fatigue Syndrome

In a study investigating the role of SIBO in CFS, 31 patients meeting the U.S. Centers for Disease Control and Prevention criteria for CFS were given a lactulose breath test (LBT). Seventeen of these CFS subjects agreed to open label antibiotic treatment with various antibiotics, including doxycycline. 14 out of 17 had successful eradication of SIBO. CFS symptoms were evaluated 7 days after the 10-day course of antibiotics.

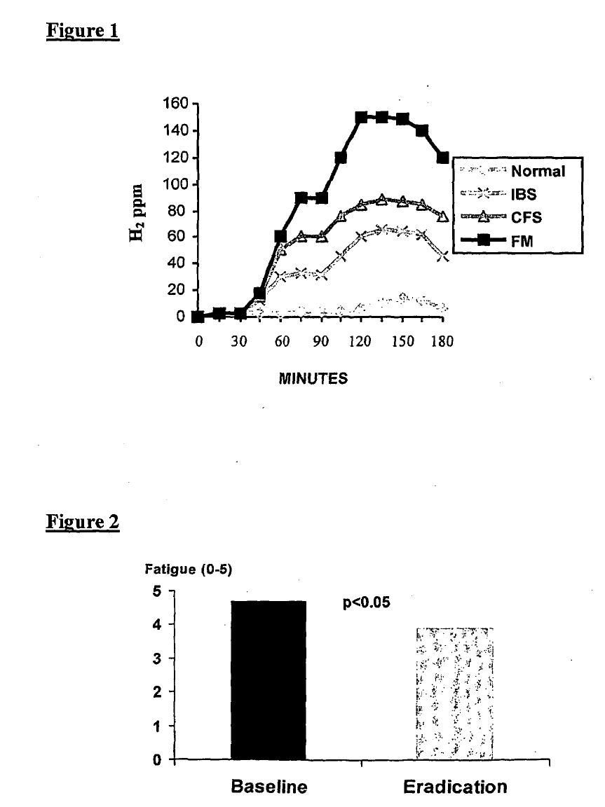

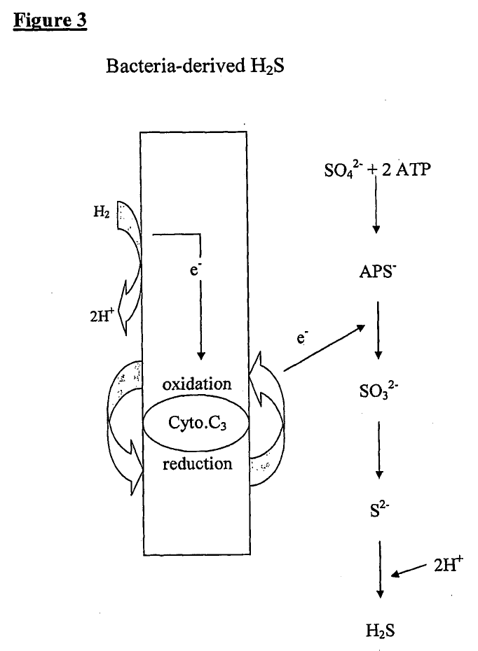

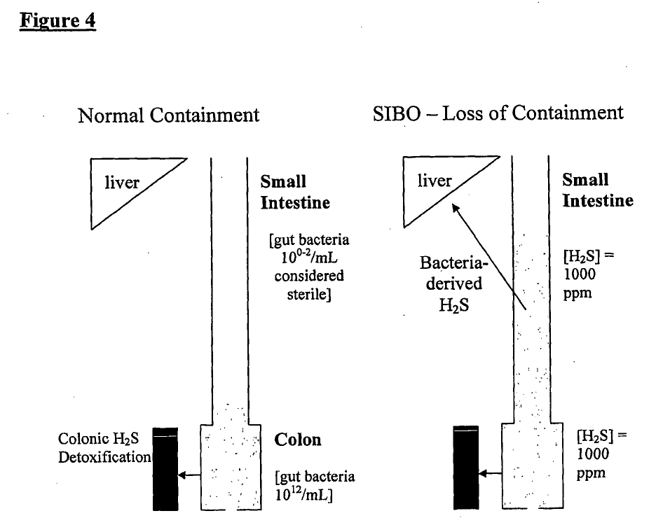

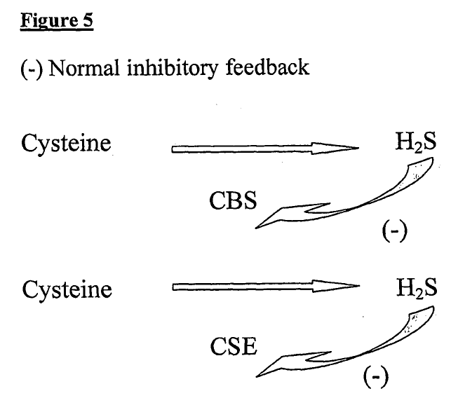

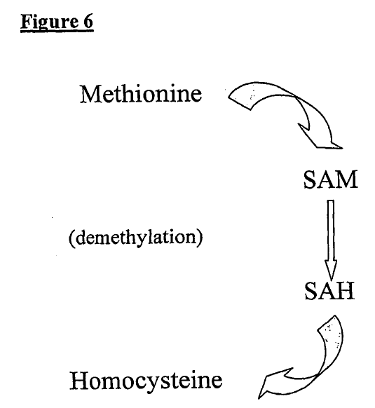

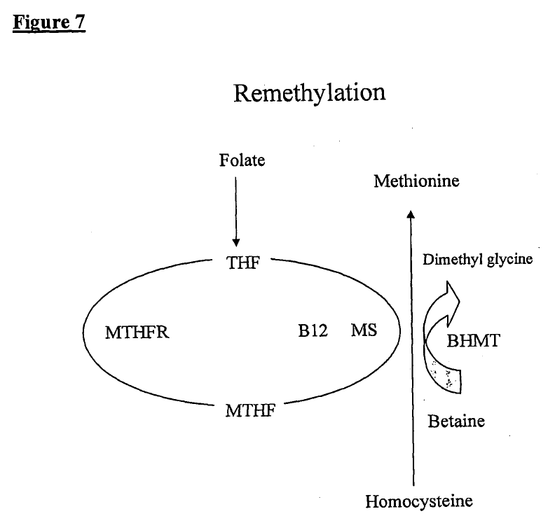

FIG. 1 shows the average breath hydrogen (H₂) profile during the LBT in CFS patients as compared to normal subjects and patients with IBS or fibromyalgia (FM). CFS patients had a peak H₂ concentration [H₂] of 85 ppm. No measurements were made of methane or H₂S in these studies. Symptom score for fatigue was rated on a scale of 0-5. Fatigue was significantly improved by eradication of SIBO (p<0.05) (FIG. 2). Bloating and gas also improved with eradication. In addition, as shown in Table 1, significant improvement was seen in the Visual Analogue Scale (VAS) scores for pain and memory/concentration (p<0.05).

TABLE 1

| | Baseline | Eradication | p-value |
|---|---|---|---|
| VAS: Pain | 74.6 ± 30.3 | 57.5 ± 27.5 | <0.05 |
| VAS: Memory/Concentration | 91.4 ± 22.4 | 66.4 ± 31.5 | <0.01 |

These studies demonstrate a high prevalence of SIBO in CFS patients, and preliminary data from the open label study showed a modest but statistically significant improvement in some symptoms. Because the second observation time point was only 7 days after completion of treatment, the full extent of improvement with time was not studied.

In a separate study, an elevated homocysteine level was found in the cerebrospinal fluid of 11 out of 11 CFS patients (B. Regland et al., Increased concentrations of homocysteine in the cerebrospinal fluid in patients with fibromyalgia and chronic fatigue syndrome, Scandinavian J. Rheumatol., 26(4):301-307 (1997)). This suggests the possibility of a block in one of the two pathways for the metabolism of homocysteine, as described above. Although only two of these 11 CFS patients had an elevated plasma level of homocysteine, this measurement was made in the fasted state (i.e., no methionine loading). This is significant, because an abnormality in the transsulfuration pathway involving CBS is often revealed only after methionine loading (N. P. Dudman et al., Disordered methionine/homocysteine metabolism in premature vascular disease. Its occurrence, cofactor therapy, and enzymology., Arterioscler. & Thromb., 13(9): 1253-1260 (1993)). Because endogenous H₂S is produced by the CBS enzyme, it is believed that exposure to bacteria-derived H₂S may interfere with this enzyme and lead to hyperhomocysteinemia especially after a methionine load.

Considerable overlap exists between the clinical presentations of CFS and that of other common functional disorders such as IBS and FM (A. J. Barsky et al., Functional somatic syndromes, Ann. Intern. Med., 130(11):910-921 (1999)). A greater degree of fatigue is reported by FM patients than either healthy subjects or chronic pain patients (J. A. Suhr, Neuropsychological impairment in fibromyalgia: relation to depression, fatigue, and pain, J. Psychosomatic Res., 55(4):321-329 (2003)). Fatigue is also identified as one of the top four problems by patients with IBS (E. Wong et al., Development of a questionnaire to measure quality of life in patients with irritable bowel syndrome, Euro. J. Surg. Acta Chirurgica, Supp., 1998(583):50-56 (1998)). With patients reporting so many of the same symptoms, they are often assigned more than one functional diagnosis so that up to 70% of patients with CFS meet the criteria for FM on the basis of their shared symptoms of myalgia and arthralgia (D. L. Goldenberg et al., High frequency of fibromyalgia in patients with chronic fatigue seen in a primary care practice, Arth. & Rheum. 33(3):381-387 (1990) and D. Buchwald et al., Comparison of patients with chronic fatigue syndrome, fibromyalgia, and multiple chemical sensitivities, Archives of Int. Med., 154(18):2049-2053 (1994)) and up to 80% of FM patients have CFS (L. A. Aaron et al., Overlapping conditions among patients with chronic fatigue syndrome, fibromyalgia, and temporomandibular disorder, Archives of Int. Med., 160(2):221-227 (2000)). In fact, fatigue and gastrointestinal symptoms are so widely shared that up to 92% of CFS patients have IBS (Goldenberg et al and L. A. Aaron et al.). Similarly, up to 80% of FM patients have IBS (A. D. Sperber et al., Fibromyalgia in the irritable bowel syndrome: studies of prevalence and clinical implications, Amer. J. Gastro. 94(12):3541-3546 (1999) and D. Veale et al., Primary fibromyalgia and the irritable bowel syndrome: different expressions of a common pathogenetic process, British J. Rheumatol., 30(3):220-222 (1991)). This suggests that a unifying explanation (i.e., SIBO) may provide a framework for understanding all of these symptoms.

CFS may be treated by at least partially eradicating bacteria overgrowth in the patient. To accomplish this, a patient is administered an agent that at least partially eradicates the bacteria overgrowth. For example, the patient is administered a quantity of an antimicrobial agent; for example an antibiotic such as rifaximin. About 400 mg to about 600 mg of rifaximin may be administered TID for about 10 days The antimicrobial agent at least partially eradicates the bacteria in the small bowel, thereby reducing the number of sulfur-reducing bacteria, hence reducing the level of bacteria-derived H₂S. The reduction of bacteria-derived H₂S results in a lower level of H₂S escaping into the blood stream. Accordingly, bacteria-derived H₂S does not interfere with the CBS enzyme and thus treats hyperhomocysteinemia and CFS.

### Example 2

### Diseases and Conditions Associated with Cognitive Impairment

As the aged population has increased, the number of elderly people with varying stages of cognitive impairment has also increased. The impairment varies from Alzheimer's disease (AD) where orientation can be severely disturbed, to those with mild loss of memory. Mild cognitive impairment (MCI) and age-associated memory impairment (AAMI) are terms used to describe individuals who suffer from cognitive impairment but are capable of functioning normally in their daily lives. MCI individuals are at increased risk to develop AD. The cause of each of these cognitive impairments was heretofore unknown.

Reversible MCI has been observed in patients recovering from infection or those with chronic inflammatory diseases raising the possibility that inflammation may be an important cause of cognitive impairment. It has been shown that SIBO is among the explanations, if not the only explanation for IBS (H. C. Lin, Small intestinal bacterial overgrowth: a framework for understanding irritable bowel syndrome, J. Amer. Med. Assoc., 292(7):852-858 (2004) and M. Pimentel et al., Eradication of small intestinal bacterial overgrowth reduces symptoms of irritable bowel syndrome, Amer. J. Gastro. 95(12):3503-3506 (2000)). IBS patients suffer from many extra-intestinal symptoms including MCI affecting concentration or short term memory. The possibility of an overlap between SIBO and MCI/AAMI populations is further supported by a high prevalence of SIBO in the elderly.

It is well known that patients recovering from infections or those diagnosed with inflammatory diseases exhibit so-called “sickness behavior.” Normal individuals who are administered cytokines systemically such as TNF-α display sickness behavior traits, such as impaired cognition, lethargy, decreased learning, and reduced mobility. Acute phase proteins are consistently found in patients with AD to support the role of inflammation. Additionally, increased levels of IL-6 and TNF-α have been found in patients with mild or moderate late-onset AD. In a recent randomized, placebo-controlled study of 100 patients, doxycycline and rifampin were shown to reduce cognitive worsening in mild to moderate AD patients (M. B. Loeb et al., A randomized, controlled trial of doxycycline and rifampin for patients with Alzheimer's disease, J. Amer. Geriatrics Soc., 52(3):381-387 (2004)). Although the target of the antibiotic treatment was not identified, this observation supports the existence of an antibiotic-sensitive, reversible bacterial mechanism. Furthermore, although these observations have been drawn primarily from AD patients, similar evidence of increased immune activity is available in MCI patients (C. J. Wilson et al., Cytokines and cognition—the case for a head-to-toe inflammatory paradigm, J. Amer. Geriatrics Soc., 50(12):2041-2056 (2002)). However, the underlying cause of this chronic inflammation was not known heretofore.

The possibility of a gastrointestinal mechanism to account for extra-intestinal manifestations such as impaired cognition was suggested by the inventor's recent studies in patients with IBS and FM. It was found that SIBO explained 84% of IBS patients and 100% of FM patients, and that successful eradication of SIBO with an antibiotic improved both gastrointestinal and extra-intestinal symptoms. The extra-intestinal symptoms may be caused by cytokines that are released as a part of the immune response to bacterial translocation (i.e., movement of gut bacteria from the lumen across the mucosal barrier), which is a known complication of SIBO (R. D. Berg et al, Immunosuppression and intestinal bacterial overgrowth synergistically promote bacterial translocation, Arch. of Surg. 123(11): 1359-1364 (1988) and V. B. Nieuwenhuijs et al., The role of interdigestive small bowel motility in the regulation of gut microflora, bacterial overgrowth, and bacterial translocation in rats, Ann. Surg., 228(2):188-193 (1998)).

Moreover, homocysteine has been studied extensively in cardiovascular disease, and has recently crossed over to the field of cognitive disorders due to growing evidence demonstrating an association between cerebrovascular disease and dementia (J. A. Rhodin et al., A vascular connection to Alzheimer's disease, Microcirc., 8(4):207-220 (2001); R. Leboeuf, Homocysteine and Alzheimer's disease, J. Amer. Dietetic Assoc., 103(3):304-307 (2003); and G. C. Roman et al., Subcortical ischaemic vascular dementia, Lancet Neurol., 1(7):426-436 (2002)). Indeed, a study recently reported that elevated plasma homocysteine is an important risk factor for AD (S. Seshadri et al, Plasma homocysteine as a risk factor for dementia and Alzheimer's disease, New England J. Med., 346(7):476-483 (2002)). As described above, the biochemistry involved in homocysteine cycling provides a clue to a possible link with H₂S (i.e., exposure to high levels of exogenous H₂S may inhibit CBS, leading to decreased production of endogenous H₂S that is important for cognitive ability). Indeed, low brain H₂S levels and increased homocysteine levels have both been observed in AD patients (K. Eto et al., Hydrogen sulfide is produced in response to neuronal excitation, J. Neurosci., 22(9):3386-3391 (2002)).

AD, MCI and AAMI may also be treated by at least partially eradicating bacteria overgrowth in the patient. To accomplish this, a patient is administered an agent that at least partially eradicates the bacteria overgrowth. For example, the patient is administered a quantity of a probiotic agent; for example B. infantis (e.g., Align®, available from Proctor & Gamble) to be taken as one capsule in the morning. The probiotic agent at least partially eradicates the bacteria in the small bowel, thereby reducing the number of sulfur-reducing bacteria, hence reducing the level of bacteria-derived H₂S. The reduction of bacteria-derived H₂S results in a lower level of H₂S escaping into the blood stream. Accordingly, bacteria-derived H₂S does not decrease the level of activity of the CBS enzyme in the brain and thus treats AD, MCI and AAMI.

### Example 3

### Relationship Among Vascular/Heart Disease and Hyperhomocysteinemia-Induced DNA and Protein Hypomethylation with Therapeutic Lowering of Homocysteine Level

A 25% reduction of homocysteine level or drop of 3 μM/L is associated with an 11% lowering of the risk of ischemic heart disease and a 19% lowering of the risk of stroke (The Homocysteine Studies Collaboration, Homocysteine and risk of ischmic heart disease and stroke: a meta-analysis, J. Amer. Med. Assoc., 288:2015-22 (2002)). While some have explained this relationship by the effects of homocysteine including increased oxidative stress, enhanced coagulation, decreased fibrinolysis and impaired endothelial biology, another possibility is that elevated homocysteine increases the levels of S-adenosylmethionine (SAM), which is indirectly harmful because it inhibits transmethylation—SAM is a potent inhibitor of transmethylation (C. van Guldener et al., Hyperhomocysteinaemia and vascular disease—a role for DNA hypomethylation, Lancet, 361:1668-1669 (2003)).

Moreover, reduced methylation (including DNA and protein hypomethylation) may alter gene expression, leading to abnormal vascular biology, enhanced lipid deposition, defective membrane repair, inhibition of endothelial cell growth, and accelerated atherosclerosis (M. Oshizumi et al., Inhibition of growth and p21ras methylation in vascular endothelial cells by homocysteine but not cysteine, J. Biol. Chem., 272:25380-25385 (1997)), and A. F. Perna et al., Enzymatic methyl esterification of erythrocyte membrane proteins is impaired in chronic renal failure: evidence for high levels of natural inhibitor of S-adenosylhomocysteine, J. Clin. Invest., 91:2497-503 (1993)).

It is therefore believed that the diagnosis and treatment of SIBO/bacteria-derived H₂S may be linked with hyperhomocysteinemia-induced DNA and protein hypomethylation via increase in SAM. Therefore, in various embodiments of the present invention, diseases and conditions pathologically associated with hyperhomocysteinemia-induced DNA and protein hypomethylation may be treated by at least partially eradicating SIBO, and can be diagnosed and/or monitored by studying SIBO levels (e.g., by lactulose breath test). In addition, bacteria-derived H₂S may be specifically detected in the exhaled breath using gas analyzers sensitive to sulfur-containing compounds.

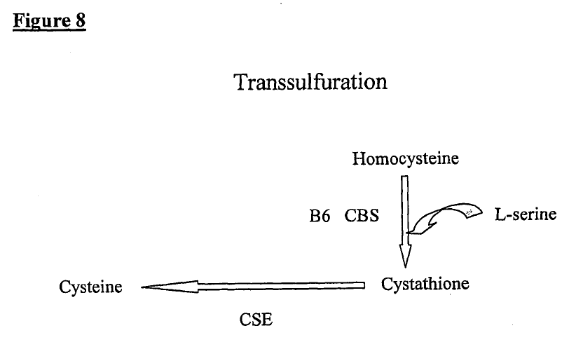

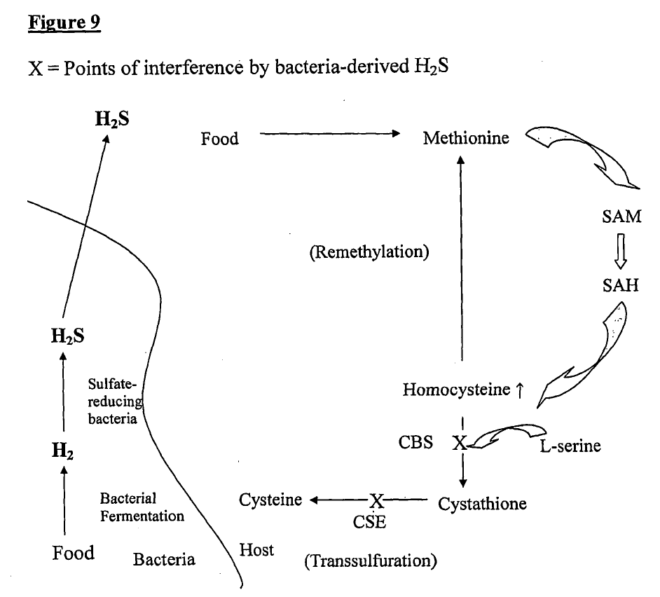

Vascular/heart disease may also be treated by at least partially eradicating bacteria overgrowth in the patient. To accomplish this, a patient is administered an agent that at least partially eradicates the bacteria overgrowth. For example, the patient is administered a quantity of a 4-aminosalicylate compound; for example, 4(p)-aminosalicylic acid (i.e., mesalamine). About 800 mg of Asacol® (i.e., mesalamine) may be administered TID for about 6 weeks. Another mesalamine compound, Pentasa®, may be administered at the dose of 1 g four times a day (QID) for about 8 weeks. The 4-aminosalicylate compound at least partially eradicates the bacteria in the small bowel, thereby reducing the number of sulfur-reducing bacteria, hence reducing the level of bacteria-derived H₂S. The reduction of bacteria-derived H₂S results in a lower level of H₂S escaping into the blood stream. Accordingly, bacteria-derived H₂S does not interfere with the CBS enzyme, thus resulting in a reduced level of homocysteine. (See FIG. 8.) The reduced level of homocysteine also reduces the level of SAM, and thus reduces the inhibition of transmethylation, thus treating vascular/heart disease by reducing the cardiovascular complications associated with hyperhomocysteinemia.

### Example 4

### Targeting Energy Transfer of Sulfate-Reducing Bacteria

Sulfate-reducing bacteria are a special group of anaerobic bacteria that derive their oxidative metabolism not from fermentation but rather from the reduction of sulfate or certain other inorganic sulfur compounds. These organisms require alkaline pH for survival. Reduction of sulfate to sulfide involves three enzymes, ATP sulfurylase, pyrophosphatase and APS reductase (D. A. Ware et al., Nature (Lond.), 226:1250). Reduction of sulfite to sulfide involves a six-electron transfer process (W. Nakatsukasa et al., J. Bact., 98:429 (1969)), whereby:

3H₂+SO₃²⁻+S²⁻+3H₂O and 2 enzymes (thiosulfate reductase and sulfide reductase)

These sulfur-reducing bacteria belong to the spore-forming genus Desulfotomaculum (L. I. Campbell et al., Bact. Rev., 29:359 (1965)) and Desulfovibrio (J. R. Postgate et al., Bact. Rev. 39:732 (1966)). Iron is necessary in the oxidation of hydrogen by these organisms (J. C. Sadana et al., Archs. Biochem. Biophys., 108:255 (1964)), because this micronutrient is involved in electron transport and phosphorylation involving cytochromes types C3, b and d as well as ferrodoxin, rubredoxin and flavodoxin (J. M. Akagi, J. Biol. Chem., 242:2478 (1967)). Bacterial ferrodoxin bear homology to ferrodoxins in green plants and is therefore different than mammalian ferrodoxin.

Therefore, in an embodiment of the invention, the sulfur-reducing bacteria can be at least partially eradicated and/or their physiological impact of their host mitigated by several methods; for instance, administering to the patient a therapeutic agent that binds iron in the intestine (e.g., lactoferrin) or administering an iron competing agent (e.g., bismuth (Pepto-bismol)); by administering a therapeutic agent or compound that releases oxygen in the intestine (e.g., sodium dihydrogen orthophosphate); and/or by administering a therapeutic agent or that decreases pH within the intestine (e.g., vitamin C). Alternatively, treatment strategies can be readily implemented to target bacterial, and not mammalian ferrodoxin in vivo. In addition, by administering electron acceptors such as nitrates, zinc, iron, barite and bismuth (Mitsui T, Edmond L M, Magee E A, Cummings J H. The effects of bismuth, iron, zinc and nitrates on free sulfide in batch cultures seeded with fecal flora. Clinica Chimica Acta 335:131-135, 2003), bacteria-derived H₂S may be reduced via blockade of the energy transfer needed for the growth of sulfate-reducing bacteria.

### Example 5

### Relationship Among Headaches including Migraines and Therapeutic Lowering of H₂S Level

Migraine is a vascular headache of unknown etiology treated with vasoconstrictors such as sumatriptan (M. Lainez, Clinical benefits of early triptan therapy for migraine, Cephalagia, 24 Supp. 2:24-30 (2004)). One-year prevalence of migraine in Sweden was reported to be 13.2±1.9% of the population (M. Linde et al., Attitudes and burden of disease among self-considered migraineurs—a nation-wide population-based survey in Sweden, Cephalagia, 24(6):455-65 (2004)). While in experimental animal model cerebral vasodilation may be induced with inhalation of carbon dioxide (M. Fukuda et al., Effects of sumatriptan on cerebral blood flow under normo- and hypercapnia in rats, Cephalagia, 22(6):468-473 (2002)), the mechanism for painful dilatation of cerebral vessels in migraine is not known. One of the biologic effects of H₂S is relaxation of vascular smooth muscle (W. Zhao et al., The vasorelaxant effect of H(2)S as a novel endogenous gaseous K(ATP) channel opener, EMBO Journal, 20(21):6008-6016 (2001)). While this has been shown to decrease blood pressure, it is believed that exposure to bacteria-derived H₂S may be responsible for the vasodilatation of cerebral blood vessels in migraine. Migraine has also been reported to be associated with hyperhomocysteinemia (F. DiSabato, STRESSEN in the treatment of psycho-physical stress and hyperhomocysteinemia in patients with migraine without aura, Clinica Terapeutica, 155(1):21-3 (2004), B. Grubler, Migraine, inflammation, genes. New risk factors for stroke, MMW Fortschritte der Medizin, 145(51-52):10 (2003)). There is a significant association between migraine and ischemic stroke (R. Z. Kern, Progress in clinical neurosciences: Migraine-stroke: a causal relationship, but which direction?, Can. J. Neurol. Sci. 31(40):451-459 (2004)) as well as migraine and cardiac disease (G. Pierangeli et al., The role of cardiac diseases in the comorbidity between migraine and stroke, Neurol. Sci., 25 Supp. 3:S129-31 (2004)). It is not clear, however, whether one leads to the other or if these co-morbid conditions share a common underlying mechanism. It is believed that the common mechanism may be SIBO, the resultant immune activation, and/or the adverse effects of bacteria-derived H₂S including hyperhomocysteinemia. It is further believed that the management of migraine may include detection of and treatment of SIBO, H₂S excretion and/or hyperhomocysteinemia as described above.

### Example 6

### Hepatopulmonary Syndrome (HPS)

A hypoxic condition encountered in patients with the following triad-1. Arterial deoxygenation: alveolar-arterial oxygen tension (AaO₂) difference of >15 mmHg, 2. Intrapulmonary vascular dilatation (IPVD) as demonstrated by a positive contrast-enhanced echocardiography (CEE) where micro air bubbles injected into an arm vein appear in left atrium and 3. Liver disease with or without cirrhosis.

Etiology of HPS is not known. Findings in humans include: endotoxemia (Guarner C, Soriano G, Tomas A, Bulbena O, Novella M T, Balanzo J, Vilardell F, Mourelle M, Moncada S. Increased serum nitrite and nitrate levels in patients with cirrhosis: relationship to endotoxemia. Hepatology 1993; 18:1139-1143) and hyperkinetic circulatory state with abnormally low systemic and pulmonary vascular resistance. Possible role of NO as suggested by a case report of response to L-NAME in a single pt. with HPS who had enhanced oxygenation and was able to walk an extra 92M distance (Brussino L, Bucca C, Morello M, Scappaticci E, Mauro M, Rolla G. Effect on dyspnoea and hypoxaemia of inhaled N(G)-nitro-L-arginine methyl ester in hepatopulmonary syndrome. Lancet 2003; 362:43-44).

HPS is reproduced in rat model of chronic bile duct ligation as rats with 5-6 weeks of chronic bile duct ligation leading to biliary cirrhosis exhibit changes similar to HPS including: 1. Increased cardiac output, decreased total systemic and pulmonary vascular resistance when compared to sham-operated animals, 2. Cirrhosis, 3. Poor alveolar gas exchange as evidenced by increased A-a O₂ and 4. Increased intrapulmonary shunting (increased arterial recovery of microspheres injected into femoral vein) (Chang S W, O'Hara N. Pulmonary circulatory dysfunction in rats with biliary cirrhosis. An animal model of the hepatopulmonary syndrome. Am Rev Respir Dis 1992; 145:798-805) as demonstrated by greater shunt with 5 weeks vs. 2 weeks of common bile duct ligation (Fallon M B, Abrams G A, McGrath J W, Hou Z, Luo B. Common bile duct ligation in the rat: a model of intrapulmonary vasodilatation and hepatopulmonary syndrome. Am J Physiol 272:G779, 1997). Other findings in the rat model of HPS include: 1. Increased heme oxygenase-1 expression and CO production (Carter E P, et al. Am J Physiol Lung Cell Mol Physiol 2002; 283:L346-L353), 2. Increased pulmonary iNOS and eNOS expression (Fallon M B, Abrams G A, Luo B, Hou Z, Dai J, Ku D D. The role of endothelial nitric oxide synthase in the pathogenesis of a rat model of hepatopulmonary syndrome Gastroenterology 1997; 113:606-614), 3. L-NAME improved but did not correct hypoxia (Zhang X J, Katsuta Y, Akimoto T, Ohsuga M, Aramaki T, Takano T. Intrapulmonary vascular dilatation and nitric oxide in hypoxemic rats with chronic bile duct ligation. J Hepatol 2003; 39:724-730), 3. Increased endothelin- and ET-B receptor expression leading to increased pulmonary eNOS (Luo B, Abrams G A, Fallon M B. Endothelin-1 in the rat bile duct ligation model of hepatopulmonary syndrome: correlation with pulmonary dysfunction. J Hepatol 1998; 29:571-578).

A role for gut bacteria is also suggested when Norfloxacin treatment normalized iNOS expression with severity of intrapulmonary vascular dilatation reduced by about ½ (Rabiller A, Nunes H, Lebrec D, Tazi K A, Wartski M, Dulmet E, Libert J M, Mougeot C, Moreau R, Mazmanian M, Humbert M, Herve P. Prevention of gram-negative translocation reduces the severity of hepatopulmonary syndrome. Am J Respir Crit. Care Med 2002; 166(4):514-517).

Bile duct ligation animal models are well known for another defect as interdigestive (fasting) motility pattern or major migratory complex (MMC) is abnormal with reduced frequency of this specialized motility pattern which is also known as the intestinal housekeeper wave (Li Y F, Newton T J, Weisbrodt N W, Moody F G. Intestinal migrating myoelectric complexes in rats with acute pancreatitis and bile duct ligation. J Surg Res 55:182-187, 1993). This association is due to the link between bile acids and MMC as the frequency of MMC was reduced 48 and 72 h after common bile duct ligation in rats. Similarly, Biliary diversion in dogs resulted in loss of MMC and loss of motilin cycling; both restored with ursodeoxycholic acid iv (Kajiyama Y, Irie M, Enjoji A, Ozeki K, Ura K, Kanematsu T. Role of bile acids in duodenal migrating motor complexes in dogs. Dig Dis Sci 1998; 43(10):, 2278-83).

The function of MMC is to contain gut bacteria to the distal end of the gastrointestinal tract. Humans are colonized by bacteria within 24 h of birth. By the 3rd to 4th week of life, ˜100 trillion bacteria set up residence in the digestive tract. Levitt reported in 1969 the remarkable finding that the gut bacteria are compartmentalized to the distal end of this open tube organ so that the concentration reaches as high as 10¹²/ml in the colon but crossing the ileocecal valve, the concentration drops to only 10³/ml in the proximal ileum and nearly sterile in the more proximal regions of the small intestine (Levitt Md. Production and excretion of hydrogen gas in man. N Engl J Med 281:122, 1969). Keeping the 100 trillion indigenous gut bacteria contained in the distal portion of the gut (colon and distal small intestine) is an important function of the MMC. When MMC is absent or reduced in frequency, small intestinal bacterial overgrowth (SIBO) is a consequence (Vantrappen G, Janssens J, Hellemans J, Ghoos Y. The interdigestive motor complex of normal subjects and patients with bacterial overgrowth of the small intestine. J Clin Invest 1977; 59(6):1158-66).

The link between MMC and SIBO is relevant to cirrhotic patients as a reduced frequency of MMC and SIBO are both found in cirrhotic patients with history of spontaneous bacterial peritonitis (Chang C S, Chen G H, Lien H C, Yeh H Z. Small intestine dysmotility and bacterial overgrowth in cirrhotic patients with spontaneous bacterial peritonitis. Hepatology 1998; 28:1187-1190). Stool-type bacteria found in the jejunal juice of 55.6% of 27 alcoholics vs. 15.4% of 13 hospitalized controls (p<0.025). >10[5] CFU concentration of bacteria found in 48.1% of alcoholics vs. 7.6% of hospitalized controls (p<0.001) (Bode J C, Bode C, Heidelbach R, Durr H K, Martini G A. Jejunal microflora in patients with chronic alcohol abuse. Hepatogastroenterology 1984; 31(1):30-4). 59% of 53 pts with cirrhosis had SIBO by jejunal culture (>10[5] CFU/mL); SIBO was significantly correlated with endotoxemia (Bauer T M, Schwacha H, Steinbruckner B, Brinkmann F E, Ditzen A K, Aponte J J, Pelz K, Berger D, Kist M, Blum H E. Small intestinal bacterial overgrowth in human cirrhosis is associated with systemic endotoxemia. Am J Gastroenterol 2002; 97:2364-2370).

In the setting of SIBO, there is expansion of the site of bacterial fermentation into the small intestine. H₂S along with other gases are generated in this region, a region not well equipped to detoxify this toxic gas. Hydrogen is produced by gut bacteria during fermentation of food. Hydrogen is converted to either methane by methanogens or reduced to hydrogen sulfide by sulfate reducing bacteria. H₂S is normally rapidly absorbed by colon and detoxified by conversion to nonvolatile thiosulfate (Levitt M D, Fume J, Springfield J, Suarez F, DeMaster E. Detoxification of hydrogen sulfide and methanethiol in the cecal mucosa. J Clin Invest. 1999; 104(8): 1107-17). Although colonic detoxification system for H₂S is uniquely effective, the small intestine is not similarly equipped (detoxification capacity of the colon >20× that of ileum) (Fume J, Springfield J, Koenig T, DeMaster E, Levitt M D. Oxidation of hydrogen sulfide and methanethiol to thiosulfate by rat tissues: a specialized function of the colonic mucosa. Biochem Pharm 2001; 62(2):255-9). In the setting of SIBO, there would be 2 sources of H₂S: endogenous and exogenous or bacteria-derived H₂S.

The physiologic effects of H₂S in the cardiovascular system include 1. reduction in blood pressure in rats by opening ATP-dependent potassium channels, 2. Relaxation of vasculature by H2S is directed at smooth muscle cells and 3. Vasoactive effects of H₂S is not reduced by inhibitor of soluble guanylate cyclase (suggesting that the relaxatory effect is not dependent on NO) (Zhao W, Zhang J, Lu Y, Wang R. The vasorelaxant effect of H(₂)S as a novel endogenous gaseous K(ATP) channel opener. EMBO J. 2001; 20:6008-6016).

Since the luminal concentration of H₂S reaches 3000 microM within the large intestine, the small intestine would be exposed to this high concentration of H₂S in the setting of SIBO where the bacterial fermentation would take place in both the small and large intestine. Such high amounts of bacteria-derived H₂S could then be exerting supra-physiologic effects leading to vasodilatation in both systemic and pulmonary circulatory systems and the findings of hepatopulmonary syndrome.

### Example 7

### Hydrogen Sulfide May Cause Slowing of Intestinal Transit

Hydrogen sulfide (H₂S) is a highly toxic gas generated in the GI tract by indigenous gut bacteria. H₂S concentration can reach a concentration of 0.1 mM in the stomach and 3 mM in the colon, the normal site of gut bacterial flora. In the setting of small intestinal bacterial overgrowth, the small bowel as well as the colon may be exposed to this gas. Although H₂S is known as a relaxant of the smooth muscles of the gastrointestinal tract (Hosoki R, Matsuki N, Kimura H. The possible role of hydrogen sulfide as an endogenous smooth muscle relaxant in synergy with nitric oxide. Biochem Biophys Res Commun 237:527-531, 1997; Teague B, Asiedu S, Moore P K. The smooth muscle relaxant effect of hydrogen sulphide in vitro:evidence for the physiological role to control intestinal contractility. Br J Pharmacol 137:139-145. 2002), it was not known whether H₂S slows intestinal transit.

The inventor determined the effect of hydrogen sulfide on intestinal transit. Five dogs were equipped with duodenal and midgut fistulas. With occluding Foley catheters in the distal limb of the 2 fistulas, the small intestine was compartmentalized into the proximal and distal ½ of the gut. The proximal gut was perfused with pH 7.0 phosphate buffer with 0 or 1 mM NaHS (a donor of H₂S) at 2 ml/min while the distal gut was perfused with pH 7.0 phosphate buffer with 0 or 60 mM oleate at 2 ml/min for 90 minutes. 60 mM oleate was used to trigger the ileal brake response to slow intestinal transit. Intestinal transit across the proximal gut was measured by the cumulative % recovery of a radioactive marker out of the midgut fistula during the last 30 min of the 90-min perfusion (data=mean±SE).

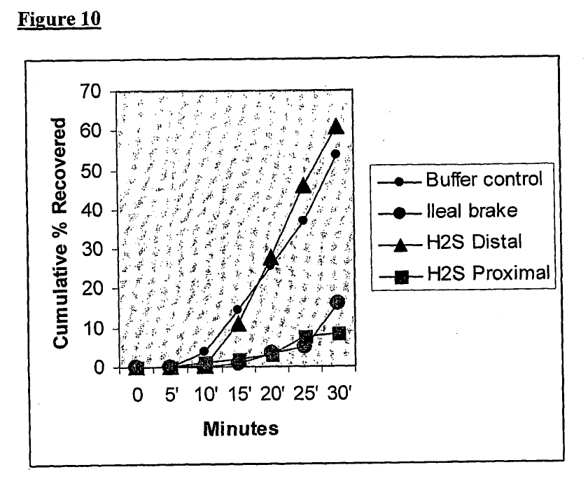

Intestinal transit was slowed by fat in the distal ½ of gut as the ileal brake response (Buffer control: 53.77±5.96% vs. Ileal brake: 16.00±3.92%) (p<0.002). Hydrogen sulfide perfused in proximal compartment (H₂S Proximal) slowed transit when compared to Buffer control (37.78±3.80% vs. 53.77±5.96%) (p<0.016). Hydrogen sulfide perfused in distal compartment (H₂S Distal) did not slow transit when compared to Buffer control (60.72±8.97% vs. 53.77±5.96%) (p<0.57). Thus, hydrogen sulfide slowed transit when perfused in the proximal compartment, but not the distal. (See FIG. 10.)

### Example 8

### Restless Legs Syndrome (RLS)

RLS is considered a movement disorder characterized by leg discomforts that are relieved with movement. The deep-seated sensation is described by patients with the use of such terms as “achy, heavy, sore, crawling paresthesia, tingling, crampy, itching, need to move” (Ondo W, Jankovic J. Restless legs syndrome: clinicoetiologic correlates. Neurology 47:1435-1441, 1996) These discomforts may occur throughout the day but are often more frequent in the evenings. The patient describes shaking their legs in a rhythmic fashion, almost at times out of their awareness. They would be told by family members or friends “to stop shaking”. In contrast to deliberate exercises using the same limbs, in RLS, the shaking can be sustained without much fatigue. Disturbed sleep for either patients or their bed partners as a result of the leg movements is a complaint affecting about 80% of patients with RLS (Montplaisir J, Boucher S, Poirier G, Lavigne G, Lapierre D, Lesperance P. Clinical, polysomnographic, and genetic characteristics of restless legs syndrome: a study of 133 patients diagnosed with new standard criteria. Mov Disord 12: 61-65, 1997). RLS is a common condition estimated to affect 3 to 9% of the general population (Ohayon M M, Roth T. Prevalence of restless legs syndrome and periodic limb movement in the general population. J Psychosom Res 53:547-554, 2002; Egan D, O'Dubhghaill C, McNamee S, Mulkerrin E, O'Keefe S T. A community study of the prevalence of restless legs. Ir Med J 96:153, 2003; Ulfberg J, Nystrom B, Carter N, Eding C. Prevalence of restless legs syndrome among men aged 18 to 64 years: an association with somatic disease and neuropsychiatric symptoms. Mov Disord 16:1159-1163, 2001; Phillips B, Young T, Finn L, Asher K, Hening W A, Purvis C. Epidemiology of the restless legs syndrome in adults. Arch Intern Med 160:2137-2141, 2000). The diagnosis of RLS is based on meeting clinical criteria established by a NIH workshop involving the International RLS Study Group. There are 4 criteria that are critical to the diagnosis: (1) An urge to move the legs, usually accompanied or caused by uncomfortable and unpleasant sensations in the legs; (2) Unpleasant sensations that begins or worsens during periods of rest or inactivity, such as lying or sitting; (3) Unpleasant sensations that are partially or totally relieved by movement; and (4) Unpleasant sensations that are worse in the evening or at night (Allen R P, Picchietti D, Hening W A, Trenkwalder C, Walters A S, Montplaisi J. Restless legs syndrome: diagnostic criteria, special considerations and epidemiology. A report from the restless legs syndrome diagnosis and epidemiology workshop at the National Institutes of Health. Sleep Med 4:101-119, 2003). The cause of RLS is unknown although dysfunction of the dopaminergic pathways is suspected and has led to the use of dopamine receptor acting agents (Happe S, Trenkwalder C. Role of dopamine receptor agonists in the treatment of restless legs syndrome. CNS Drugs. 2004; 18(1):27-36) including levodopa (Garcia-Borreguero D, Larrosa O, Granizo J J, de la Llave Y, Hening W A. Circadian variation in neuroendocrine response to L-dopa in patients with restless legs syndrome. Sleep. 2004 Jun. 15; 27(4):669-73), pramipexole (Kushida C A. Pramipexole for the treatment of restless legs syndrome. Expert Opin Pharmacother. 2006 March; 7(4):441-51) and ropinirole (Jost W H. Ropinirole: current status of the studies. J. Neurol. 2004 Sep. 251 Suppl 6:VI/13-8). Although some RLS patients have iron deficiency anemia, iron replacement therapy was not found to be effective in reducing symptoms of RLS in a randomized, placebo-controlled study (Ekhom K A. Restless legs syndrome. Acta Med Scand 158(suppl): 1-123, 1945). Exposure of exercising volunteers to 10 ppm H₂S resulted in rise of blood lactate (Bhambhani Y, Burnham R, Snydmiller G, MacLean I. Effects of 10-ppm hydrogen sulfide inhalation in exercising men and women. Cardiovascular, metabolic and biochemical responses. J Occup Environ Med 1997; 39:122-9).). RLS is one of the overlap syndromes with many patients having symptoms meeting the criteria for IBS and fibromyalgia (FM) (Matallana L, Bradley L A, Silverman S, Yunus M B. Fibromyalgia: Treating overlapping conditions. The Complete Idiot's Guide to Fibromyalgia. Chapter 13) RLS occurs in 31% of patients with FM. RLS patients also have heightened pin-prick sensitivity by a factor of 5.3 to suggest that hypersensitivity is an important clinical profile for these patients (Stiasny-Kolster K, Magerl W, Oertel W H, Moller J C, Treede R D. Static mechanical hyperalgesia without dynamic tactile allodynia in patients with restless legs syndrome. Brain. 2005 Jun, 128(Pt 6):E34). The inventor believes that since IBS and FM are hypersensitivity disorders associated with small intestinal bacterial overgrowth (SIBO) (Lin H C. Small intestinal bacterial overgrowth: a framework for understanding irritable bowel syndrome. JAMA. 2004 Aug. 18; 292(7):852-8; Pimentel M, Wallace D, Hallegua D, Chow E, Kong Y, Park S, Lin H C. A link between irritable bowel syndrome and fibromyalgia may be related to findings on lactulose breath testing. Ann Rheum Dis. 2004 Apr, 63(4):450-2; Pimentel M, Chow E J, Lin H C. Normalization of lactulose breath testing correlates with symptom improvement in irritable bowel syndrome. a double-blind, randomized, placebo-controlled study. Am J. Gastroenterol. 2003 Feb, 98(2):412-9), exposure to bacteria-derived H₂S may be the link between RLS, IBS and FM. Since H₂S production is likely to be greater after a larger meal such as the dinner meal rather than breakfast, it would be consistent with the predominant evening timing of RLS symptoms. Since H₂S exposure is associated with a shift of skeletal muscle metabolism from aerobic to anaerobic with build-up of tissue lactic acid, both the sensation of the achy discomfort and the improvement with movement of the legs can be explained. As such, it is believed that treatment of RLS can by accomplished by reducing or eliminating bacteria-derived H₂S by detecting and treating small bowel intestinal overgrowth and H₂S excretion as described above.

### Example 9

### Interstitial Cystitis as a Hypersensitivity Disorder

Interstitial cystitis (IC) is a painful bladder condition (Gillenwater J Y, Wein A J. Summary of the National Institute of Arthritis, Diabetes, Digestive and Kidney Diseases workshop on interstitial cystitis, National Institutes of Health, Bethesda, Md., August 28-29, 1987. J Urology 140:203-206) that describes patients with symptoms of dysuria, increased urinary frequency, decreased voiding volume, suprapubic pain and pressure discomfort accompanied by urinary urgency, dyspareunia, a sensation of incomplete voiding of the bladder and nocturia whose urine cultures are unremarkable. Many patients also undergo cystoscopy and/or cytometric studies with no explanation found for the symptoms. While some mucosal findings such as ulcers or linear cracks, known as Hunner's ulcers (Hunner G L. A rare type of bladder ulcers in women: report of cases. Boston Med Surg J 172:660, 1915) that are associated with inflammatory infiltrates, particularly of T-cells, have been observed in rare patients, the majority of patients do not have any visible abnormality on inspection of the bladder mucosa. Since increased number of mast cells in either mucosal or muscular layers have also been reported, IC is considered a disorder with pathophysiology involving activated immunity whereby increased tissue levels of proinflammatory mediators and neurotransmitters including substance P, histamine, interleukins, CGRP, VIP and chemokines may result in vasodilatation, activation of afferent nerves and recruitment of inflammatory cells. Using 48 voiding diaries, IC patients have been found to have objective findings for bladder hypersensitivity as documented by increased frequency of voiding and decreased volume of urine flow (van Ophoven A, Rossbach G, Oberpenning F, Hertle L. Hyperbaric oxygen for the treatment of interstitial cystitis: Long-term results of a prospective pilot study. Eur Urology 45; 108-113, 2004). Since the cause of IC was heretofore unknown, there is no generally agreed upon or effective treatment for this condition with treatments varying from transcutaneous electrical nerve stimulation and transurethral resection to pentosanpolysulfate administered either orally or by injection to subcutaneous treatments with heparin (Peeker R, Fall M. Treatment guidelines for classic and non-ulcer interstitial cystitis. Int Urogycol J Pelvic Floor Dysfunction 11(1):w23-32, 2000; Rovner E, Propert K J, Brensinger C, Wein A J, Foy M, Kirkemo A, et al. Treatments used in women with interstitial cystitis: the interstitial cystitis data base (ICDB) study experience. The Interstitial Cystitis Data Base Study Group. Urology 56(6):940-945, 2000; Propert K J, Schaeffer A J, Brensinger C M, Kusek J W, Nyberg L M, Landis J R. A prospective study of interstitial cystitis: results of longitudinal follup of the interstitial cystitits data base cohort. The Interstitital Cystitits Data Base Study Group. J Urol 163(5): 1434-1439, 2000; Fall M, Johansson S I, Aldenborg F. Chronic interstitial cystitis: a heterogeneous syndrome. J Urol 137:35, 1987). IC patients commonly have symptoms meeting the clinical criteria for other overlap disorders that have been grouped under the term central sensitivity syndrome including IBS, fibromyalgia and chronic fatigue syndrome. Indeed, fatigue is a prominent complaint of these patients (Messing E M, Stamey T A. Interstitial cystitis: early diagnosis, pathology and treatment. Urology 12:381, 1978). Since IBS and FM are associated with a high prevalence of small intestinal bacterial overgrowth, bacteria-derived H₂S may provide another mechanism to explain the hypersensitivity. The glutamate receptor, N-methyl-D-aspartate (NMDA) receptor is involved in the transmission of painful sensation. In a rat model of visceral hypersensitivity as monitored by response to colorectal distension, central and peripherally administered NMDA receptor antagonist succeeded in blocking the nociception (Gaudreau G A, Plourde V. Involvement of N-methyl-D-aspartate (NMDA) receptor in a rat model of visceral hypersensitivity. Behav Brain Res 150(1-2):185-9, 2004). NMDA receptor is now shown to have a critical role in visceral hypersensitivity in humans (Willert R P, Woolf C J, Hobson, A R, Delaney C, Thompson D G, Aziz Q. The development and maintenance of human visceral pain hypersensitivity is dependent on the N-methyl-D-aspartate receptor. Gastroenterology 126(3):683-692, 2000). Hyperalgesia and allodynia are now explained on the basis of excitatory amino acids causing a state of hyperexcitation of NMDA receptors with accompanied reduction in input from inhibitory interneuron to result in a longer lasting response to painful stimulus known as long-term potentiation on long term depression (Amantea B, Gemelli A, Militano D, Salatino I, Caroleo S, Neuronal plasticity and neuropathic pain. Minerva Anestesiol 66(12):901-911). Since at physiologic concentrations, H₂S has been identified to potentiate the NMDA (N-methyl-D-aspartate) receptor-mediated responses (Kimura H. Hydrogen sulfide induces cyclic AMP and modulates the NMDA receptor. Biochem Biophys Res Commun 267:129-133, 2000), exposure to bacteria-derived H₂S would provide a mechanism for maintenance of a hypersensitivity state as in interstitial cystitis. In addition, the stimulatory effect of H₂S on afferent nerves of the bladder is supported by the finding that H₂S stimulates contractions of urinary bladder muscles via a neurogenic mechanism involving capsaicin-sensitive primary afferent nerves equipped with transient receptor vanilloid-1 receptors (TRPV1) and efferent nerves acting on tachykinin 1 and tachykinin 2 receptors rather than acting via K_{ATP} channels (Patacchini R, Santicioli P, Giuliani S, Maggi C A. Hydrogen sulfide (H2S) stimulates capsaicin-sensitive primary afferent neurons in the rat urinary bladder. Br J Pharmacol 142:31-34, 2004; Patacchini R, Santicioli P, Giuliani S, Maggi C A. Pharmacological investigation of the hydrogen sulfide (H₂S) contractile activity in rat detrusor muscle. Eur J Pharmacol 509:171-177, 2005; Trevisani M, Patacchini R, Nicoletti P, et al. Br J Pharm 145(8): 1123-32, 2005). Furthermore, increased sensitivity of the skin was reported by workers exposed to carbon disulphide (Takebayashi T, Omae K, Ishizuka C, Nomiyama T, Sakurai H. Cross sectional observation of the effects of carbon disulphide on the nervous system, endocrine system, and subjective symptoms in rayon manufacturing workers. Occup Environ Med. 1998 Jul, 55(7):473-9). The inventor believes that H₂S-mediated sensitization provides an explanation for hypersensitivity disorders such as IC, FM, headaches, IBS, etc. Accordingly, the detection and treatment of bacteria-derived H₂S can result in the treatment of IC, FM, headaches, IBS, etc.

### Example 10

### Rats with Experimental SIBO, Thiosulfate and H₂S Measurements

Thiosulfate concentration in portal blood and H₂S concentration in peripheral blood may be greater in SIBO. The inventor measured collected portal blood and systemic blood from a control rat and 3 rats with experimentally induced SIBO. SIBO was induced by delivering into the stomach, by gavage, a slurry containing 24% raw red kidney beans twice a day for 10 days. On this feeding, the small intestine becomes colonized by colonic bacteria within 24 h (Banwell et al. “Bacterial overgrowth by indigenous microflora in the phytohemagglutinin-fed rat,” Can J. Microbiol. (1988), 34:1009-1013; Banwell et al. “Intestinal microbial flora after feeding phytohemagglutinin lectins (Phaseolus vulgaris) to rats,” Appl Environ Microbiol. (1985) 50(1):68-80). On day 10, the rats were euthanized 3 h after 0.5 ml lactulose, as a fermentable substrate, was delivered into the stomach by gavage. Thiosulfate was measured by HPLC using Model 5600 CoulArray electrochemical detector (ESA, Inc. Chelmsford, Mass.)(Rebrin et al. “Effects of age and caloric restriction on glutathione redox state in mice,” Free Rad Biol Med. (2003) 35: 626-635). Plasma H₂S concentration was measured by spectrophotometry.

Results of portal blood thiosulfate concentration in microCoul (μC): Red Kidney Bean fed rats (SIBO): 21.0 μC, 72.1 μC, 19.9 μC vs. Control rat (no gavage): 6.2 μC. Standard values were represented by y=3.4304x+15.641, R2=0.98 where x=μM, y=μC. Results of systemic blood H₂S concentration in μM: Red Kidney Bean fed rats (SIBO): 90.9 μM, 100.1 μM, 99.9 μM vs. Control rat (no gavage): 10.6 μM.

The concentration of portal thiosulfate and H₂S were elevated in a rat model of SIBO to provide support for increased H₂S exposure in SIBO.

### Example 11

### Preparation for Detection of H₂S or Thiosulfate

One day prior to the test, subjects are instructed to eat a light dinner with rice as the starch and to refrain from eating after midnight. Testing should be done in the morning. One blood sample is drawn in the fasted state for measurement of homocysteine, H₂S and thiosulfate. Then a quantity of lactulose (e.g., 10 g) is given. Alternatively, lactulose and methionine (e.g., L-methionine) are given. To promote bacterial H₂S production, drive homocysteine production and interfere with transsulfuration all at the same time, lactulose and methionine are administered together.

### Example 12

### Lactulose Breath Test

Lactulose breath test is performed before, 2 hours and 4 hours after a subject ingests a quantity of lactulose or lactulose and methionine. Alternatively, breath samples can also be intermittently collected for three to four hours. The presence of H₂S is diagnostic of CFS in a patient. H₂S concentration in exhaled breath may be measured using a total/species sulfur analyzer.

Additionally, in patients having at least one symptom associated with a suspected diagnosis of a disease condition related to bacteria-derived H₂S, the presence and/or concentration of H₂S in the breath corroborates with the diagnosis and/or the prognosis of the disease condition.

### Example 13

### H₂S Measurement in Blood

Blood samples are collected 2 hours and 4 hours after the ingestion of the lactulose or the lactulose and methionine for measurement of thiosulfate and/or H₂S. 500 μl of plasma is collected and 375 μl of 1% zinc acetate is added immediately to trap the H₂S. H₂S may also be measured in blood and blood cells. Plasma is stored for a maximum of 24 hours before performing the colorimetric assay for H₂S as follows: 350 μl of the plasma-zinc acetate solution is added to a microcentrifuge tube with 620 μl of de-ionized water, 100 μl of 20 mM N,N-dimethyl-p-phenylenediamine sulfate in 7.2 M HCl and 133 μl of 30 mM FeCl3 in 1.2 M HCl. The tube is incubated in the dark for 5-10 minutes. 300 μl of 10% trichloroacetic acid is added to precipitate proteins, and samples are spun down. The absorbance of the supernatant at 650 nm is measured and compared to a standard curve of NaHS, a donor of H₂S, ranging from 10 μM to 250 μM. As an additional method for measuring hydrogen sulfide level in plasma, a sulfide-sensitive electrode (model 96-16, Orion Research) is used to measure sulfide by titration with lead perchlorate.

In patients having at least one symptom associated with a suspected diagnosis of a disease condition related to bacteria-derived H₂S, the presence and/or concentration of H₂S in the blood corroborates with the diagnosis and/or the prognosis of the disease condition.

### Example 14

### Thiosulfate Measurements in Blood and/or Urine

Thiosulfate is measured by reverse-phase ion-pair high performance liquid chromatography (HPLC). This procedure may be used for either blood or urine samples. Urine samples are collected at the time the blood samples are collected. Monbromobimane is used for precolumn derivatization. This method takes advantage of the property of this substance to yield fluorescent compounds upon reaction with thiosulfate. This method has accuracy to as low as 0.16 mmol.

In patients having at least one symptom associated with a suspected diagnosis of a disease condition related to bacteria-derived H₂S, the presence and/or concentration of thiosulfate in the blood and/or urine corroborates with the diagnosis and/or the prognosis of the disease condition.

While the description above refers to particular embodiments of the present invention, it should be readily apparent to people of ordinary skill in the art that a number of modifications may be made without departing from the spirit thereof. The presently disclosed embodiments are, therefore, to be considered in all respects as illustrative and not restrictive, the scope of the invention being indicated by the appended claims rather than the foregoing description. All changes that come within the meaning of and range of equivalency of the claims are intended to be embraced therein.

What is claimed is:

1. A method for treating a disease condition in a mammal with an elevated level of bacteria-derived hydrogen sulfide (H₂S), comprising:
providing a therapeutic agent capable of at least partially eradicating the bacterial overgrowth to reduce the level of bacteria-derived H₂S in the mammal; and
administering the therapeutic agent to the mammal,
wherein the disease condition is a disease condition related to a category selected from the group consisting of hypercoagulable states related to hyperhomocysteinemia, vasodilatatory states, interference with function as neurotransmitter, interference with endocrine function, chronic pain syndromes due to stimulation of N-methyl-D-aspartate (NMDA) receptors leading to hypersensitivity, injury to nasal and/or respiratory tract, interference with visceral smooth muscle contractile function, inhibition of aerobic metabolism/ischemia disorders, triggering of inflammation, overlap disorders, interference with regulation of apoptosis and proliferation and combinations thereof.

2. The method of claim 1, wherein the disease condition is selected from the group consisting of hyperhomocysteinemia, chronic renal failure, end stage renal disease, hemodialysis, peritoneal dialysis, vascular dementia, cardiovascular disease, stroke, cerebrovascular accidents, thrombotic disorder, hypercoagulable states, venous thrombosis, deep vein thrombosis, thrombophlebitis, thromboembolic disease, ischemic stroke, restenosis after percutaneous transluminal coronary angioplasty (PTCA), preeclampsia, vasculitis, digital ischemia, multifocal osteonecrosis, retinal vein occlusion, glaucoma, miscarriage, pregnancy complication, placental abruption, transplantation, diabetic retinopathy, ischemic bowel disease, cerebral vein thrombosis, atherosclerosis, coronary artery disease, penile venous thrombosis, impotence, central venous thrombosis, peripheral artery disease, intermittent claudication, hemorrhagic colitis, radiation enteritis, radiation colitis, visceral ischemia, acute mesenteric ischemia, chronic mesenteric ischemia, hypertension, microangiopathy, macroangiopathy, recurrent leg ulcer, carotid stenosis, occlusive vascular disease, arterial aneurysm, abdominal aortic aneurysm, congestive heart failure, hepatopulmonary syndrome, high flow state associated with chronic liver disease, migraine headache, vascular headache, dizziness, lightheadedness, orthostatic intolerance, postural hypotension, postural hypotension, postural orthostatic tachycardia syndrome, idiopathic pulmonary fibrosis, pulmonary hypertension, angioedema, vaso-vagal faints, neuroleptic malignant syndrome, learning disorder, learning disability, insomnia, dementia, age associated memory impairment, attention deficit/hyperactivity disorder (ADHD), mild cognitive impairment, Alzheimer's disease, Down's syndrome, autism, Parkinson's disease, depression, anxiety or anxiety disorder, Asperger syndrome, glucose intolerance, diabetes, reactive hypoglycemia, metabolic syndrome, low cortisol, hypothalamus-pituitary-adrenal dysfunction, myasthenia gravis syndrome, osteoporosis, autoimmune polyendocrine syndrome, chronic fatigue syndrome (CFS), central sensitivity syndrome, angina, syndrome X, chronic neck pain syndrome, chronic neuromuscular pain, osteoarthritis, muscle tension headache, chronic headache, cluster headache, temporalis tendonitis, sinusitis, atypical facial pain, trigeminal neuralgia, facial and neck pain syndrome, temperomandibular joint syndrome, idiopathic chronic low back pain, endometriosis, painful abdominal adhesions, chronic abdominal pain syndrome, coccydynia, pelvic floor myalgia (levator ani spasm), polymyositis, postherpetic neuralgia, polyradiculoneuropathies, mononeuritis multiplex, reflex sympathetic dystrophy, neuropathic pain, vulvar vestibulitis, vulvodynia, chronic regional pain syndrome, osteoarthritis, fibrositis, chronic visceral pain syndrome, female urethral syndrome, painful diverticular disease, functional dyspepsia, nonulcer dyspepsia, non-erosive esophageal reflux disease, acid-sensitive esophagus, interstitial cystitis, chronic pelvic pain syndrome, chronic urethral syndrome, chronic prostatitis, primary dysmenorrheal, dyspareunia, premenstrual syndrome (PMS), vulvodynia, ovarian remnant syndrome, ovulatory pain, pelvic congestion syndrome, myofasical pain syndrome, fibromyalgia polymyalgia rheumatica, Reiter's syndrome (reactive arthritis), rheumatoid arthritis, spondyloarthropathy, functional somatic syndromes, chronic regional pain syndromes, post polio syndrome, functional somatic syndrome, rhinitis, asthma, multiple chemical sensitivity syndrome, reactive airway dysfunction syndrome, dysnomia, sick building syndrome, asthma, idiopathic pulmonary fibrosis, idiopathic pulmonary hypertension, dysphagia, gastroparesis, functional diarrhea, chronic constipation, defecation dysfunction, dysuria, atonic bladder, neurogenic bladder, irritable bowel syndrome (IBS), ileus, chronic idiopathic pseudoobstruction, Ogilvie's syndrome, restless leg syndrome, immune dysfunction syndrome, multiple sclerosis (MS), eczema, psoriasis, atopic dermatitis, dermatitis, Crohn's disease, ulcerative colitis, ulcerative proctitis, pouchitis, nonspecific ulcerative colitis, inflammatory bowel disease (IBD), celiac disease, diversion colitis, collagenous colitis, lymphocytic colitis, blind loop syndrome, nonalcoholic steatohepatitis (NASH), fatty liver, chronic liver disease, cirrhosis, spontaneous bacterial peritonitis, postoperative ileus, systemic lupus erythematosis, mixed connective tissue disorder, undifferentiated connective tissue disorder, Raynaud's phenomenon, Kawasaki syndrome, polymyositis, dermatomyositis, myositis, multiple autoimmune syndrome, Sjögren's syndrome, lichen planus, idiopathic uveitis, gingivitis, stomatitis, otitis, necrotizing enterocolitis, intensive care unit (ICU) multiple organ failure, primary biliary cirrhosis, idiopathic myelofibrosis, polyarteritis nodosa, eosinophilic pleural effusion, eosinophilic gastroenteritis, eosinophilic esophagitis, graft vs. host disease, Grave's disease, idiopathic thyroid failure, Hashimoto's thyroiditis, autoimmune hepatitis, pancreatitis, CREST syndrome, autoimmune cholangitis, ankylosing spondylitis, atopic dermatitis, vitiligo, scleroderma, autoimmune ear disease, polyangiitis overlap syndrome, primary sclerosing cholangitis, Gulf War syndrome, myalgic encephalomyelitis, food sensitivity, dysregulation spectrum syndrome, post-traumatic stress disorder (PTSD), benign tumor, malignant tumor, cancer and combinations thereof.

3. The method of claim 1, wherein the therapeutic agent is a therapeutic agent capable of at least partially eradicating the bacterial overgrowth of sulfur-reducing bacteria.

4. The method of claim 3, wherein the therapeutic agent capable of at least partially eradicating the bacterial overgrowth of sulfur-reducing bacteria is a methanogenic bacterium that out-competes the sulfur-reducing bacteria.

5. The method of claim 1, wherein the therapeutic agent is selected from the group consisting of an antimicrobial agent, an antimicrobial chemotherapeutic agent, an intestinal lavage agent, an enema agent, a bismuth-containing compound, a compound that binds iron in the intestinal lumen, a compound that binds hydrogen sulfide, a probiotic agent, an agent that increases the mammal's phase III interdigestive intestinal motility and combinations thereof.

6. The method of claim 5, wherein the antimicrobial agent is selected from the group consisting of a natural antibiotic agent, a synthetic antibiotic agent, a semi-synthetic antibiotic agent and combinations thereof.

7. The method of claim 5, wherein the antimicrobial agent is selected from the group consisting of neomycin, metronidazole, teicoplanin, doxycycline, tetracycline, norfloxacin, ciprofloxacin, augmentin, cephalexin, penicillin, ampicillin, kanamycin, rifamycin, rifaximin, vancomycin and combinations thereof.

8. The method of claim 5, wherein the antimicrobial chemotherapeutic agent is a 4-aminosalicylate compound or a 5-aminosalicylate compound.

9. The method of claim 5, wherein the antimicrobial chemotherapeutic agent is selected from the group consisting of 4-(p)-aminosalicylic acid, 4-(p)-aminosalicylate sodium salt, 5-aminosalicylic acid, conjugated derivatives thereof, conjugated bile acids thereof and combinations thereof.

10. The method of claim 5, wherein the probiotic agent is selected from the group consisting of a Bifidobacterium species, a Lactobacillus species and combinations thereof.

11. The method of claim 5, wherein the probiotic agent is selected from the group consisting of L. acidophilus, L. rhamnosus, L. plantarum, L. reuteri, L. paracasei, L. casei Shirota, L. salivarius, B. infantis and combinations thereof.

12. The method of claim 5, wherein the agent that increases the mammal's phase III interdigestive intestinal motility is a prokinetic agent.

13. The method of claim 12, wherein the prokinetic agent is selected from the group consisting of a bile acid, a bile salt, a prokinetic peptide, a macrolide compound, a 5-hydroxytryptamine receptor directed drug, a 5-HT4 receptor agonist, a 5-HT receptor antagonist a compound with cholinergic activity, a dopamine antagonist, a nitric oxide altering agent, an antihistamine, a neuroleptic agent, a kappa agonist and combinations thereof.

14. The method of claim 12, wherein the prokinetic agent is selected from the group consisting of motilin, erythromycin, azithromycin, tegaserod, ondansetron, cilansetron, granisetron, alosetron, ursodeoxycholic acid, chenodeoxycholic acid, a salt of ursodeoxycholate, a salt of chenodeoxycholate, cisapride, metoclopramide, domperidone, bethanechol, octreotide, cholecystonin, nitroglycerin, nomega-nitro-L-arginine methylester (L-NAME), N-monomethyl-L-arginine (L-NMMA), promethazine, meclizine, prochlorperazine, chlorpromazine, haloperidol and combinations thereof.

15. A method for diagnosing and/or determining the prognosis of a disease condition related to an elevated level of bacteria-derived hydrogen sulfide (H₂S) in a mammal having at least one symptom associated with a suspected diagnosis of the disease condition, comprising:
detecting the presence and/or concentration of hydrogen sulfide (H₂S) and/or thiosulfate; and
diagnosing and/or determining the prognosis of the disease condition,
wherein the disease condition is a disease condition related to a category selected from the group consisting of hypercoagulable states related to hyperhomocysteinemia, vasodilatatory states, interference with function as neurotransmitter, interference with endocrine function, chronic pain syndromes due to stimulation of N-methyl-D-aspartate (NMDA) receptors leading to hypersensitivity, injury to nasal and/or respiratory tract, interference with visceral smooth muscle contractile function, inhibition of aerobic metabolism/ischemia disorders, triggering of inflammation, overlap disorders interference with regulation of apoptosis and proliferation and combinations thereof.

16. The method of claim 15, wherein the disease condition is selected from the group consisting of hyperhomocysteinemia, chronic renal failure, end stage renal disease, hemodialysis, peritoneal dialysis, vascular dementia, cardiovascular disease, stroke, cerebrovascular accidents, thrombotic disorder, hypercoagulable states, venous thrombosis, deep vein thrombosis, thrombophlebitis, thromboembolic disease, ischemic stroke, restenosis after percutaneous transluminal coronary angioplasty (PTCA), preeclampsia, vasculitis, digital ischemia, multifocal osteonecrosis, retinal vein occlusion, glaucoma, miscarriage, pregnancy complication, placental abruption, transplantation, diabetic retinopathy, ischemic bowel disease, cerebral vein thrombosis, atherosclerosis, coronary artery disease, penile venous thrombosis, impotence, central venous thrombosis, peripheral artery disease, intermittent claudication, hemorrhagic colitis, radiation enteritis, radiation colitis, visceral ischemia, acute mesenteric ischemia, chronic mesenteric ischemia, hypertension, microangiopathy, macroangiopathy, recurrent leg ulcer, carotid stenosis, occlusive vascular disease, arterial aneurysm, abdominal aortic aneurysm, congestive heart failure, hepatopulmonary syndrome, high flow state associated with chronic liver disease, migraine headache, vascular headache, dizziness, lightheadedness, orthostatic intolerance, postural hypotension, postural hypotension, postural orthostatic tachycardia syndrome, idiopathic pulmonary fibrosis, pulmonary hypertension, angioedema, vaso-vagal faints, neuroleptic malignant syndrome, learning disorder, learning disability, insomnia, dementia, age associated memory impairment, attention deficit/hyperactivity disorder (ADHD), mild cognitive impairment, Alzheimer's disease, Down's syndrome, autism, Parkinson's disease, depression, anxiety or anxiety disorder, Asperger syndrome, glucose intolerance, diabetes, reactive hypoglycemia, metabolic syndrome, low cortisol, hypothalamus-pituitary-adrenal dysfunction, myasthenia gravis syndrome, osteoporosis, autoimmune polyendocrine syndrome, chronic fatigue syndrome (CFS), central sensitivity syndrome, angina, syndrome X, chronic neck pain syndrome, chronic neuromuscular pain, osteoarthritis, muscle tension headache, chronic headache, cluster headache, temporalis tendonitis, sinusitis, atypical facial pain, trigeminal neuralgia, facial and neck pain syndrome, temperomandibular joint syndrome, idiopathic chronic low back pain, endometriosis, painful abdominal adhesions, chronic abdominal pain syndrome, coccydynia, pelvic floor myalgia (levator ani spasm), polymyositis, postherpetic neuralgia, polyradiculoneuropathies, mononeuritis multiplex, reflex sympathetic dystrophy, neuropathic pain, vulvar vestibulitis, vulvodynia, chronic regional pain syndrome, osteoarthritis, fibrositis, chronic visceral pain syndrome, female urethral syndrome, painful diverticular disease, functional dyspepsia, nonulcer dyspepsia, non-erosive esophageal reflux disease, acid-sensitive esophagus, interstitial cystitis, chronic pelvic pain syndrome, chronic urethral syndrome, chronic prostatitis, primary dysmenorrheal, dyspareunia, premenstrual syndrome (PMS), vulvodynia, ovarian remnant syndrome, ovulatory pain, pelvic congestion syndrome, myofasical pain syndrome, fibromyalgia polymyalgia rheumatica, Reiter's syndrome (reactive arthritis), rheumatoid arthritis, spondyloarthropathy, functional somatic syndromes, chronic regional pain syndromes, post polio syndrome, functional somatic syndrome, rhinitis, asthma, multiple chemical sensitivity syndrome, reactive airway dysfunction syndrome, dysnomia, sick building syndrome, asthma, idiopathic pulmonary fibrosis, idiopathic pulmonary hypertension, dysphagia, gastroparesis, functional diarrhea, chronic constipation, defecation dysfunction, dysuria, atonic bladder, neurogenic bladder, irritable bowel syndrome (IBS), ileus, chronic idiopathic pseudoobstruction, Ogilvie's syndrome, restless leg syndrome, immune dysfunction syndrome, multiple sclerosis (MS), eczema, psoriasis, atopic dermatitis, dermatitis, Crohn's disease, ulcerative colitis, ulcerative proctitis, pouchitis, nonspecific ulcerative colitis, inflammatory bowel disease (IBD), celiac disease, diversion colitis, collagenous colitis, lymphocytic colitis, blind loop syndrome, nonalcoholic steatohepatitis (NASH), fatty liver, clronic liver disease, cirrhosis, spontaneous bacterial peritonitis, postoperative ileus, systemic lupus erythematosis, mixed connective tissue disorder, undifferentiated connective tissue disorder, Raynaud's phenomenon, Kawasaki syndrome, polymyositis, dermatomyositis, myositis, multiple autoimmune syndrome, Sjögren's syndrome, lichen planus, idiopathic uveitis, gingivitis, stomatitis, otitis, necrotizing enterocolitis, intensive care unit (ICU) multiple organ failure, primary biliary cirrhosis, idiopathic myelofibrosis, polyarteritis nodosa, eosinophilic pleural effusion, eosinophilic gastroenteritis, eosinophilic esophagitis, graft vs. host disease, Grave's disease, idiopathic thyroid failure, Hashimoto's thyroiditis, autoimmune hepatitis, pancreatitis, CREST syndrome, autoimmune cholangitis, ankylosing spondylitis, atopic dermatitis, vitiligo, scleroderma, autoimmune ear disease, polyangiitis overlap syndrome, primary sclerosing cholangitis, Gulf War syndrome, myalgic encephalomyelitis, food sensitivity, dysregulation spectrum syndrome, post-traumatic stress disorder (PTSD), benign tumor, malignant tumor, cancer and combinations thereof.

17. The method of claim 15, wherein the detecting the presence and/or concentration of H₂S comprises:
detecting the presence and/or concentration of H₂S in the mammal's breath and/or blood.

18. The method of claim 17, wherein the detecting the presence and/or concentration of H₂S in the mammal's breath and/or blood comprises:
providing a quantity of a poorly digestible sugar;
administering the quantity of the poorly digestible sugar to the mammal;
obtaining a breath sample and/or a blood sample from the mammal; and
analyzing the breath sample and/or the blood sample to determine the presence and/or concentration of H₂S.

19. The method of claim 18, wherein the poorly digestible sugar is selected from the group consisting of glucose, lactose, lactulose, xylose and combinations thereof.

20. The method of claim 18, wherein the analyzing the breath sample comprises:
analyzing the breath sample using a gas analyzer sensitive to sulfur or sulfur containing compounds to determine the presence and/or concentration of H₂S.

21. The method of claim 18, wherein the analyzing the breath sample comprises:
analyzing the breath sample using a total/species sulfur analyzer to determine the presence and/or concentration of H₂S.

22. The method of claim 18, wherein the analyzing the blood sample further comprises:
providing a quantity of zinc acetate; and
adding the quantity of zinc acetate to the blood sample,
whereby the quantity of zinc acetate traps the H₂S in the blood.

23. The method of claim 18, wherein the analyzing the blood sample comprises:
analyzing the blood sample using a calorimetric assay for H₂S, a sulfide-sensitive electrode or spectrophotometry.

24. The method of claim 15, wherein detecting the presence and/or concentration of thiosulfate comprises:
detecting the presence and/or concentration of thiosulfate in the mammal's blood and/or urine.

25. The method of claim 24, wherein the detecting the presence and/or concentration of thiosulfate in the mammal's blood and/or urine of the mammal comprises:
providing a quantity of a poorly digestible sugar;
administering the quantity of the poorly digestible sugar to the mammal;
obtaining a blood sample and/or a urine sample from the mammal; and
analyzing the blood sample and/or the urine sample for the presence and/or concentration of thiosulfate.

26. The method of claim 25, wherein the poorly digestible sugar is selected from the group consisting of glucose, lactose, lactulose, xylose and combinations thereof.

27. The method of claim 25, wherein the analyzing the blood sample and/or the urine sample comprises:
analyzing the blood sample and/or the urine sample for the presence and/or concentration of thiosulfate using liquid chromatography.

28. The method of claim 27, wherein the liquid chromatography is reverse-phase ion-pair high performance liquid chromatography.

29. A kit for the treating a disease condition in a mammal with an elevated level of bacteria-derived hydrogen sulfide (H₂S), comprising:
a therapeutic agent capable of at least partially eradicating bacterial overgrowth; and
instructions to administer the therapeutic agent to the mammal to reduce bacteria-derived H₂S in the mammal,
wherein the disease condition is a disease condition related to a category selected from the group consisting of hypercoagulable states related to hyperhomocysteinemia, vasodilatatory states, interference with function as neurotransmitter, interference with endocrine function, chronic pain syndromes due to stimulation of N-methyl-D-aspartate (NMDA) receptors leading to hypersensitivity, injury to nasal and/or respiratory tract, interference with visceral smooth muscle contractile function, inhibition of aerobic metabolism/ischemia disorders, triggering of inflammation, overlap disorders, interference with regulation of apoptosis and proliferation and combinations thereof.

30. The kit of claim 29, wherein the disease condition is selected from the group consisting of hyperhomocysteinemia, chronic renal failure, end stage renal disease, hemodialysis, peritoneal dialysis, vascular dementia, cardiovascular disease, stroke, cerebrovascular accidents, thrombotic disorder, hypercoagulable states, venous thrombosis, deep vein thrombosis, thrombophlebitis, thromboembolic disease, ischemic stroke, restenosis after percutaneous transluminal coronary angioplasty (PTCA), preeclampsia, vasculitis, digital ischemia, multifocal osteonecrosis, retinal vein occlusion, glaucoma, miscarriage, pregnancy complication, placental abruption, transplantation, diabetic retinopathy, ischemic bowel disease, cerebral vein thrombosis, atherosclerosis, coronary artery disease, penile venous thrombosis, impotence, central venous thrombosis, peripheral artery disease, intermittent claudication, hemorrhagic colitis, radiation enteritis, radiation colitis, visceral ischemia, acute mesenteric ischemia, chronic mesenteric ischemia, hypertension, microangiopathy, macroangiopathy, recurrent leg ulcer, carotid stenosis, occlusive vascular disease, arterial aneurysm, abdominal aortic aneurysm, congestive heart failure, hepatopulmonary syndrome, high flow state associated with chronic liver disease, migraine headache, vascular headache, dizziness, lightheadedness, orthostatic intolerance, postural hypotension, postural hypotension, postural orthostatic tachycardia syndrome, idiopathic pulmonary fibrosis, pulmonary hypertension, angioedema, vaso-vagal faints, neuroleptic malignant syndrome, learning disorder, learning disability, insomnia, dementia, age associated memory impairment, attention deficit/hyperactivity disorder (ADHD), mild cognitive impairment, Alzheimer's disease, Down's syndrome, autism, Parkinson's disease, depression, anxiety or anxiety disorder, Asperger syndrome, glucose intolerance, diabetes, reactive hypoglycemia, metabolic syndrome, low cortisol, hypothalamus-pituitary-adrenal dysfunction, myasthenia gravis syndrome, osteoporosis, autoimmune polyendocrine syndrome, chronic fatigue syndrome (CFS), central sensitivity syndrome, angina, syndrome X, chronic neck pain syndrome, chronic neuromuscular pain, osteoarthritis, muscle tension headache, chronic headache, cluster headache, temporalis tendonitis, sinusitis, atypical facial pain, trigeminal neuralgia, facial and neck pain syndrome, temperomandibular joint syndrome, idiopathic chronic low back pain, endometriosis, painful abdominal adhesions, chronic abdominal pain syndrome, coccydynia, pelvic floor myalgia (levator ani spasm), polymyositis, postherpetic neuralgia, polyradiculoneuropathies, mononeuritis multiplex, reflex sympathetic dystrophy, neuropathic pain, vulvar vestibulitis, vulvodynia, chronic regional pain syndrome, osteoarthritis, fibrositis, chronic visceral pain syndrome, female urethral syndrome, painful diverticular disease, functional dyspepsia, nonulcer dyspepsia, non-erosive esophageal reflux disease, acid-sensitive esophagus, interstitial cystitis, chronic pelvic pain syndrome, chronic urethral syndrome, chronic prostatitis, primary dysmenorrheal, dyspareunia, premenstrual syndrome (PMS), vulvodynia, ovarian remnant syndrome, ovulatory pain, pelvic congestion syndrome, myofasical pain syndrome, fibromyalgia polymyalgia rheumatica, Reiter's syndrome (reactive arthritis), rheumatoid arthritis, spondyloarthropathy, functional somatic syndromes, chronic regional pain syndromes, post polio syndrome, functional somatic syndrome, rhinitis, asthma, multiple chemical sensitivity syndrome, reactive airway dysfunction syndrome, dysnomia, sick building syndrome, asthma, idiopathic pulmonary fibrosis, idiopathic pulmonary hypertension, dysphagia, gastroparesis, functional diarrhea, chronic constipation, defecation dysfunction, dysuria, atonic bladder, neurogenic bladder, irritable bowel syndrome (IBS), ileus, chronic idiopathic pseudoobstruction, Ogilvie's syndrome, restless leg syndrome, immune dysfunction syndrome, multiple sclerosis (MS), eczema, psoriasis, atopic dermatitis, dermatitis, Crohn's disease, ulcerative colitis, ulcerative proctitis, pouchitis, nonspecific ulcerative colitis, inflammatory bowel disease (IBD), celiac disease, diversion colitis, collagenous colitis, lymphocytic colitis, blind loop syndrome, nonalcoholic steatohepatitis (NASH), fatty liver, chronic liver disease, cirrhosis, spontaneous bacterial peritonitis, postoperative ileus, systemic lupus erythematosis, mixed connective tissue disorder, undifferentiated connective tissue disorder, Raynaud's phenomenon, Kawasaki syndrome, polymyositis, dermatomyositis, myositis, multiple autoimmune syndrome, Sjögren's syndrome, lichen planus, idiopathic uveitis, gingivitis, stomatitis, otitis, necrotizing enterocolitis, intensive care unit (ICU) multiple organ failure, primary biliary cirrhosis, idiopathic myelofibrosis, polyarteritis nodosa, eosinophilic pleural effusion, eosinophilic gastroenteritis, eosinophilic esophagitis, graft vs. host disease, Grave's disease, idiopathic thyroid failure, Hashimoto's thyroiditis, autoimmune hepatitis, pancreatitis, CREST syndrome, autoimmune cholangitis, ankylosing spondylitis, atopic dermatitis, vitiligo, scleroderma, autoimmune ear disease, polyangiitis overlap syndrome, primary sclerosing cholangitis, Gulf War syndrome, myalgic encephalomyelitis, food sensitivity, dysregulation spectrum syndrome, post-traumatic stress disorder (PTSD), benign tumor, malignant tumor, cancer and combinations thereof.

31. The kit of claim 29, wherein the composition is selected from the group consisting of a methanogenic bacterium, a antimicrobial agent, an antimicrobial chemotherapeutic agent, an intestinal lavage agent, an enema agent, a bismuth-containing compound, a compound that binds iron in the intestinal lumen, a compound that binds hydrogen sulfide, a probiotic agent, a prokinetic agent and combinations thereof.

32. A kit for diagnosing and/or determining a prognosis of a disease condition related to an elevated level of bacteria-derived hydrogen sulfide (H₂S) in a mammal having at least one symptom associated with a suspected diagnosis of the disease condition, comprising:
a poorly digestible sugar; and
instructions to use the poorly digestible sugar to diagnose and/or determine a prognosis of the disease condition,
wherein the disease condition is a disease condition related to a category selected from the group consisting of hypercoagulable states related to hyperhomocysteinemia, vasodilatatory states, interference with function as neurotransmitter, interference with endocrine function, chronic pain syndromes due to stimulation of N-methyl-D-aspartate (NMDA) receptors leading to hypersensitivity, injury to nasal and/or respiratory tract, interference with visceral smooth muscle contractile function, inhibition of aerobic metabolism/ischemia disorders, triggering of inflammation, overlap disorders, interference with regulation of apoptosis and proliferation and combinations thereof.

33. The kit of claim 32, wherein the disease condition is selected from the group consisting of hyperhomocysteinemia, chronic renal failure, end stage renal disease, hemodialysis, peritoneal dialysis, vascular dementia, cardiovascular disease, stroke, cerebrovascular accidents, thrombotic disorder, hypercoagulable states, venous thrombosis, deep vein thrombosis, thrombophlebitis, thromboembolic disease, ischemic stroke, restenosis after percutaneous transluminal coronary angioplasty (PTCA), preeclampsia, vasculitis, digital ischemia, multifocal osteonecrosis, retinal vein occlusion, glaucoma, miscarriage, pregnancy complication, placental abruption, transplantation, diabetic retinopathy, ischemic bowel disease, cerebral vein thrombosis, atherosclerosis, coronary artery disease, penile venous thrombosis, impotence, central venous thrombosis, peripheral artery disease, intermittent claudication, hemorrhagic colitis, radiation enteritis, radiation colitis, visceral ischemia, acute mesenteric ischemia, chronic mesenteric ischemia, hypertension, microangiopathy, macroangiopathy, recurrent leg ulcer, carotid stenosis, occlusive vascular disease, arterial aneurysm, abdominal aortic aneurysm, congestive heart failure, hepatopulmonary syndrome, high flow state associated with chronic liver disease, migraine headache, vascular headache, dizziness, lightheadedness, orthostatic intolerance, postural hypotension, postural hypotension, postural orthostatic tachycardia syndrome, idiopathic pulmonary fibrosis, pulmonary hypertension, angioedema, vaso-vagal faints, neuroleptic malignant syndrome, learning disorder, learning disability, insomnia, dementia, age associated memory impairment, attention deficit/hyperactivity disorder (ADHD), mild cognitive impairment, Alzheimer's disease, Down's syndrome, autism, Parkinson's disease, depression, anxiety or anxiety disorder, Asperger syndrome, glucose intolerance, diabetes, reactive hypoglycemia, metabolic syndrome, low cortisol, hypothalamus-pituitary-adrenal dysfunction, myasthenia gravis syndrome, osteoporosis, autoimmune polyendocrine syndrome, chronic fatigue syndrome (CFS), central sensitivity syndrome, angina, syndrome X, chronic neck pain syndrome, chronic neuromuscular pain, osteoarthritis, muscle tension headache, chronic headache, cluster headache, temporalis tendonitis, sinusitis, atypical facial pain, trigeminal neuralgia, facial and neck pain syndrome, temperomandibular joint syndrome, idiopathic chronic low back pain, endometriosis, painful abdominal adhesions, chronic abdominal pain syndrome, coccydynia, pelvic floor myalgia (levator ani spasm), polymyositis, postherpetic neuralgia, polyradiculoneuropathies, mononeuritis multiplex, reflex sympathetic dystrophy, neuropathic pain, vulvar vestibulitis, vulvodynia, chronic regional pain syndrome, osteoarthritis, fibrositis, chronic visceral pain syndrome, female urethral syndrome, painful diverticular disease, functional dyspepsia, nonulcer dyspepsia, non-erosive esophageal reflux disease, acid-sensitive esophagus, interstitial cystitis, chronic pelvic pain syndrome, chronic urethral syndrome, chronic prostatitis, primary dysmenorrheal, dyspareunia, premenstrual syndrome (PMS), vulvodynia, ovarian remnant syndrome, ovulatory pain, pelvic congestion syndrome, myofasical pain syndrome, fibromyalgia polymyalgia rheumatica, Reiter's syndrome (reactive arthritis), rheumatoid arthritis, spondyloarthropathy, functional somatic syndromes, chronic regional pain syndromes, post polio syndrome, functional somatic syndrome, rhinitis, asthma, multiple chemical sensitivity syndrome, reactive airway dysfunction syndrome, dysnomia, sick building syndrome, asthma, idiopathic pulmonary fibrosis, idiopathic pulmonary hypertension, dysphagia, gastroparesis, functional diarrhea, chronic constipation, defecation dysfunction, dysuria, atonic bladder, neurogenic bladder, irritable bowel syndrome (IBS), ileus, chronic idiopathic pseudoobstruction, Ogilvie's syndrome, restless leg syndrome, immune dysfunction syndrome, multiple sclerosis (MS), eczema, psoriasis, atopic dermatitis, dermatitis, Crohn's disease, ulcerative colitis, ulcerative proctitis, pouchitis, nonspecific ulcerative colitis, inflammatory bowel disease (IBD), celiac disease, diversion colitis, collagenous colitis, lymphocytic colitis, blind loop syndrome, nonalcoholic steatohepatitis (NASH), fatty liver, chronic liver disease, cirrhosis, spontaneous bacterial peritonitis, postoperative ileus, systemic lupus erythematosis, mixed connective tissue disorder, undifferentiated connective tissue disorder, Raynaud's phenomenon, Kawasaki syndrome, polymyositis, dermatomyositis, myositis, multiple autoimmune syndrome, Sjögren's syndrome, lichen planus, idiopathic uveitis, gingivitis, stomatitis, otitis, necrotizing enterocolitis, intensive care unit (ICU) multiple organ failure, primary biliary cirrhosis, idiopathic myelofibrosis, polyarteritis nodosa, eosinophilic pleural effusion, eosinophilic gastroenteritis, eosinophilic esophagitis, graft vs. host disease, Grave's disease, idiopathic thyroid failure, Hashimoto's thyroiditis, autoimmune hepatitis, pancreatitis, CREST syndrome, autoimmune cholangitis, ankylosing spondylitis, atopic dermatitis, vitiligo, scleroderma, autoimmune ear disease, polyangiitis overlap syndrome, primary sclerosing cholangitis, Gulf War syndrome, myalgic encephalomyelitis, food sensitivity, dysregulation spectrum syndrome, post-traumatic stress disorder (PTSD), benign tumor, malignant tumor, cancer and combinations thereof.

34. The kit of claim 32, wherein the poorly digestible sugar is selected from the group consisting of glucose, lactose, lactulose, xylose and combinations thereof.

35. The kit of claim 32, wherein the instructions to use the poorly digestible sugar to diagnose and/or determine a prognosis of the disease condition comprise:
instructions to administer the poorly digestible sugar to the mammal;
instructions to obtain a breath, blood and/or urine sample from the mammal; and
instructions to analyze the breath, blood and/or urine sample for the presence and/or concentration of H₂S and/or thiosulfate,
wherein the presence and/or concentration of H₂S and/or thiosulfate corroborates with the diagnosis or prognosis of the suspected disease condition.
